(19) United States
(12) Patent Application Publication
(10) Pub. No.: US 2026/0279499 A1
(43) Pub. Date: Sep. 17, 2026

(54) FLOW CYTOMETRY NANOPARTICLE EVENT DETECTION AND EVALUATION FROM FLUORESCENT RESPONSE DATA

(71) Applicant: Sartorius BioAnalytical Instruments, Inc., Ann Arbor, MI (US)

(72) Inventors: Mehdi Dehghani, Medford, MA (US); Paul Keselman, Saline, MI (US)

(21) Appl. No.: 19/077,856

(22) Filed: Mar. 12, 2025

(51) Int. Cl. G16B 40/10; G01N 15/1429

(52) U.S. Cl. CPC G16B 40/10; G01N 15/1429

(57) ABSTRACT

Blank flow cytometry data is analyzed excluding a portion of background signal data values for setting a minimum signal threshold requirement. Blank flow cytometry data is analyzed for setting a minimum peak width requirement. A detection condition for identifying stained nanoparticles from analysis of flow cytometry fluorescent response data includes a minimum signal threshold requirement and a minimum peak width requirement.

### FIELD OF DISCLOSURE

This disclosure relates to flow cytometry evaluation of nanoparticles.

### BACKGROUND

Flow cytometry is an analytical technique for evaluating a fluid sample for the presence of target particles of interest. Flow cytometry involves subjecting a flow of a fluid sample to a stimulus (typically light, such as from a laser) detecting a response (typically response radiation) and analyzing the response to identify occurrences of the target particles. Response detection capabilities may include detection of one or more radiation response properties, which may include detection of one or more of light scatter properties, such as forward scatter light and/or side scatter light, and detection for one or more fluorescent emission signatures of fluorescent stains that may be added to fluid samples to fluorescently label particular features of target particles. Flow cytometry is a common technique used to evaluate for the presence of cells and other similarly sized particles, which are often of a size in a range of from 2 to 20 microns. Flow cytometers used for such applications commonly include both light scatter detection with multiple light scatter detectors to permit detection of different light scatter properties and fluorescent emission detection capabilities with multiple fluorescent emission detectors to permit detection of multiple different fluorescent emission signatures provided by different fluorescent stains. Flow cytometry evaluation systems may also combine a flow cytometer with an autosampler that is capable of automated processing of sample trays containing many fluid samples for automated delivery of the fluid samples sequentially to the flow cytometer to perform sequential flow cytometry investigations of the fluid samples. Such systems are widely used in analyzing cells and particles of similar size, and provide a convenient and cost effective technique for flow cytometry analysis of many fluid samples in a relatively short amount of time.

More recently flow cytometers have been developed with capabilities to analyze for much smaller particles into the nanoparticle size range, such as virus particles (virions), virus-like particles and extracellular vesicles, including exosomes, and other similarly sized particles. Such particles may often be in a range of from 20 nanometers to one micron in size, with particle sizes smaller than 200 microns or even smaller than 100 microns being very common. These very small biological particles are sometimes generally referred to as being of a virus size or as being virus-size particles, as they tend to be of a small size within the size range representative of virions. When evaluating fluid samples for the presence of such virus-size particles by flow cytometry, techniques and practices that work well for flow cytometry analysis of cells and similarly-sized particles often do not translate well for analysis of the much smaller virus-size particles.

Although light scatter detection becomes more difficult to employ as the size of particles decreases, various flow cytometers that have been employed for analysis of virus-size particles continue to employ modified forms of light scatter detection as the primary technique for identifying the presence of particles and then employ fluorescent staining techniques and fluorescence detection for enhanced characterization of particle attributes of the detected particles, such as for particle phenotyping.

Some flow cytometers designed for analysis of virus-size particles, however, employ only fluorescence detection from fluorescent stains for identification of the presence of particles and for performing further particle characterization of particles. Examples of such flow cytometers are the Virus Counter® 3100 flow cytometer and the Virus Counter® Plus Platform flow cytometry system, which includes an integrated autosampler. These flow cytometers process very small fluid samples at lower flow rates and use only fluorescent emission detection, without light scatter detection.

One reason that light scatter detection remains a primary particle identification technique even for flow cytometry evaluation for virus-size particles is that light scatter signals tend to be much stronger than fluorescent signals. The entire particle contributes to light scatter signals, whereas fluorescent emission signals are generated only from fluorescent stains attached to the parties and subjected to excitation radiation by a light source during flow cytometry.

There is a need for improved techniques for identifying and quantifying virus-size particles, and other nanoparticles, by flow cytometry, and particularly for use with flow cytometers relying upon fluorescent signal data for identifying the presence of particles.

### SUMMARY OF THE INVENTION

Because of the fluorescent signals from nanoparticles stained with fluorescent stains tend to be fairly weak in intensity, accurately differentiating events indicative of presence of a stained nanoparticle from background signals or other signal interferences can be difficult. Critical to accurate differentiation is setting of an event condition to distinguish temporal peaks in flow cytometry florescent response data as indicative of a particle event as opposed to a non-particle signal anomaly. The inventors have found that various techniques can be applied to evaluation of data collected by fluorescent signal detector(s) during blank sample flow cytometry (that is from flow cytometry investigation of blank fluid samples in the absence of material for particle evaluation, e.g., in the absence of a biological material sample or synthetic material that could contain nanoparticles for evaluation).

One technique identified by the inventors is to exclude a portion of the blank flow cytometry data including the largest signal magnitudes from analysis of the blank flow cytometry data for background signal characteristics used to set a minimum signal threshold requirement for a detection condition. Another technique identified by the inventors is to use the blank flow cytometry data to determine a minimum peak signal width requirement that can be included as a second requirement for an event condition in combination with a minimum signal threshold requirement. It has been found that including both a minimum signal threshold requirement and a minimum peak width requirement for an event condition applied to flow cytometry fluorescent response data to identify detection events indicative of stained nanoparticles can significantly improve accuracy of distinguishing particle events and significantly improve accuracy of counts of stained nanoparticle occurrences and quantified concentration of nanoparticles in the original material in the fluid sample that was the focus of the flow cytometry investigation.

It has also been found that careful selection of a combination of a minimum signal threshold requirement and minimum peak width requirement for an event condition that, when applied to the full blank flow cytometry data, results in only a very small number of false positive detection events in the full blank flow cytometry data can both significantly improve accuracy of the analysis of flow cytometry fluorescent response data of stained fluid samples for stained nanoparticles and can reduce, and in some cases possibly eliminate, a need to routinely run blank fluid samples along with flow cytometry investigations of stained fluid samples and correct flow cytometry particle counts by subtracting corresponding blank fluid sample particle counts.

Some aspects of the present disclosure will now be summarized.

In a first aspect, the present disclosure provides a method for evaluating blank flow cytometry data for setting at least one requirement of, and optionally all requirements of, an event condition to identify as detection events temporal peaks in fluorescent response data from flow cytometry investigation of a stained fluid sample, containing a sample of material for evaluation for the presence of nanoparticles, for stained nanoparticles comprising the nanoparticles stained with a fluorescent stain providing a fluorescent response to excitation radiation during the flow cytometry investigation. The method can comprise analyzing a portion of blank flow cytometry data, wherein the portion of the blank flow cytometry data does not include at least some of the largest background signal data values, and preferably does not include some or all of a largest 10 percent (top decile) of the background signal data values.

In a second aspect, the present disclosure provides a method for determining flow cytometry background signal characteristics for setting at least one requirement of, and optionally all requirements of, an event condition to identify as detection events temporal peaks in fluorescent response data from flow cytometry investigation of a stained fluid sample, containing a sample of material for evaluation for the presence of nanoparticles, for stained nanoparticles comprising the nanoparticles stained with a fluorescent stain providing a fluorescent response to excitation radiation during the flow cytometry investigation. The method can comprise determining characteristics of a portion of blank flow cytometry data, wherein the portion of the blank flow cytometry data does not include at least some of the largest background signal data values, and preferably does not include some or all of a largest 10 percent (top decile) of the background signal data values.

In a third aspect, the present disclosure provides a method for setting at least one requirement of, and optionally all requirements of, an event condition for identifying as detection events temporal peaks in fluorescent response data from flow cytometry investigation of a stained fluid sample, containing a sample of material for evaluation for the presence of nanoparticles, for stained nanoparticles comprising the nanoparticles stained with a fluorescent stain providing a fluorescent response to excitation radiation during the flow cytometry investigation. The method can comprise setting a minimum signal threshold requirement based on characteristics of a portion of blank flow cytometry data, wherein the portion of the blank flow cytometry data does not include at least some of the largest background signal data values, and preferably does not include some or all of a largest 10 percent (top decile) of the background signal data values.

In a fourth aspect, the present disclosure provides a method for setting at least one requirement of, and optionally all requirements of, an event condition for identifying as detection events temporal peaks in flow cytometry fluorescent response data from flow cytometry investigation of a sample of material in a stained fluid sample, containing a sample of material for evaluation for the presence of nanoparticles, for stained nanoparticles comprising the nanoparticles stained with a fluorescent stain providing a fluorescent response to excitation radiation during the flow cytometry investigation. The method can comprise:
analyzing blank flow cytometry data and setting the requirements of the event condition to include, based on the analyzing:
a minimum signal threshold requirement for the fluorescent response data values; and
a minimum peak width requirement for a temporal width of the temporal peak, the minimum peak width requirement preferably comprising a plurality of the fluorescent response data values each satisfying the minimum signal threshold requirement.

In a fifth aspect, the present disclosure provides a method for evaluating a stained fluid sample, containing a sample of material for evaluation for the presence of nanoparticles, for stained nanoparticles comprising the nanoparticles stained with a fluorescent stain providing a fluorescent response to excitation radiation during the flow cytometry investigation. The method can comprise:
analyzing fluorescent response data from a flow cytometry investigation of the stained fluid sample with excitation radiation, the fluorescent response data comprising a time series of fluorescent response data values corresponding to detected magnitudes for the fluorescent response during the flow cytometry investigation; and
the analyzing the fluorescent response data comprising identifying as detection events in the time series of fluorescent response data values temporal peaks each satisfying an event condition, wherein the event condition comprises a minimum peak width requirement for a temporal width of the temporal peak and the minimum peak width requirement includes a plurality of the fluorescent response data values each satisfying a minimum signal threshold requirement.

In a sixth aspect, the present disclosure provides a method for evaluating a stained fluid sample, containing a sample of material for evaluation for the presence of nanoparticles, for stained nanoparticles comprising the nanoparticles stained with a fluorescent stain providing a fluorescent response to excitation radiation during the flow cytometry investigation. The method can comprise:
analyzing fluorescent response data from a flow cytometry investigation of the stained fluid sample with excitation radiation, the fluorescent response data comprising a time series of fluorescent response data values corresponding to detected magnitudes for the fluorescent response during the flow cytometry investigation; and
the analyzing the fluorescent response data comprising identifying as detection events in the time series of fluorescent response data values temporal peaks each satisfying an event condition, wherein the event condition comprises at least one, and preferably a plurality, of the fluorescent response data values satisfying a minimum signal threshold requirement;
and wherein: the minimum signal threshold requirement is based on characteristics of a portion of blank flow cytometry data from blank sample flow cytometry investigation with the excitation radiation of at least one blank fluid sample not including (being in the absence of) the stained nanoparticles, the blank flow cytometry data comprising background signal values corresponding to detected signal magnitudes from detection for the fluorescent response during the blank sample flow cytometry investigation; and the portion of the blank flow cytometry data not including (being in the absence of) an excluded portion of the background signal data values comprising some of the largest background signal data values, and preferably the excluded portion comprises some or all of a largest 10 percent (top decile) of the background signal data values.

In a seventh aspect, the present disclosure provides a flow cytometry data analysis system. The flow cytometry data analysis system can comprise:
at least one processor;
one or more computer-readable media, optionally being or comprising one or more non-transitory computer-readable media, having stored instructions; and
a communication connection between the at least one processor and the one or more computer-readable media, the communication connection configured for the at least one processor to access the one or more computer-readable media and the stored instructions;
wherein, the stored instructions are executable by the at least one processor to configure the at least one processor to perform as a computer-implemented method a flow cytometry data analysis operation. Some example flow cytometry data analysis operations for computer-implementation comprise:
analyzing fluorescent response data, for example according to the fifth aspect or the sixth aspect; and/or
analyzing blank flow cytometry data or a portion thereof, for example according to the first aspect or the fourth aspect; and/or
determining characteristics of a portion of blank flow cytometry data, for example according to the second aspect; and/or
setting at least one requirement, and optionally all requirements of, an event condition, for example according to the third aspect or the fourth aspect; or combinations thereof.

In an eighth aspect, the present disclosure provides a flow cytometry system, comprising:
a flow cytometry investigation system; and
a data evaluation and control system communicatively connected to the flow cytometry investigation system and configured to control operation of the flow cytometry investigation system to perform flow cytometry investigations of fluid samples. The data evaluation and control system can be configured to perform various computer-implemented methods, for example computer implementation of various flow cytometry data analysis operations. The data evaluation and control system can include the flow cytometry data analysis system of the seventh aspect, and/or can be configured to perform any of the computer-implemented methods that may be performed by the flow cytometry data analysis system of the second aspect.

Various other feature refinements and additional features are applicable to each of these and other aspects of this disclosure, as disclosed in the description below (including in the numbered Example Implementation Combinations), the figures and the appended claims. These feature refinements and additional features may be used individually or in any combination within the subject matter of the aspects summarized above or other aspects disclosed herein. Any such feature refinement or additional feature may be, but is not required to be, used with any other feature or a combination of features disclosed herein.

In various implementations, the material for evaluation by flow cytometry investigation may include a biological material suspected of containing nanoparticles, optionally biological nanoparticles. In various other implementations, the material for evaluation by flow cytometry evaluation may be suspected of containing synthetic nanoparticles.

Various of the methods described herein can be computer-implemented. In various aspects of the present disclosure, one or more computer-readable media can have stored computer-executable instructions to perform a computer-implemented method.

Other aspects of the present disclosure, and various refinements and additional features applicable to various aspects of the present disclosure, are described below and illustrated in the drawings.

### BRIEF DESCRIPTION OF DRAWINGS

It is to be understood that the drawings, and in light of the description, are provided to aid in the understanding of the various aspects and features of the present disclosure. The drawings, and features within the drawings, are not necessarily to scale and are not intended to be detailed in every respect of the illustrated features. Features illustrated in the drawings can be combined with other features, whether described herein or not.

FIG. 1 illustrates a simple example of performing a flow cytometry investigation using a single laser source for excitation radiation in a flow cytometry system in which a stained fluid sample to be investigated is hydrodynamically focused with sheath fluid.

FIG. 2 illustrates an example of partial componentry of a flow cytometer during performance of a flow cytometry investigation of a stained fluid sample stained with two different fluorescent stains each providing a different fluorescent emission signature from stained particles.

FIG. 3 illustrates an example of a temporal peak on a time series plot of voltage magnitudes of fluorescent response data values versus time.

FIG. 4 illustrates a first graphical plot of a raw data histogram of a one second interval of blank flow cytometry data from blank sample flow cytometry investigation of a clean buffered solution vs. time and a second graphical plot of the raw time series data that has been sorted by data value magnitude and plotted from smallest to largest data magnitude with a curve fitted to the plotted data value magnitudes.

FIG. 5 illustrates a first graphical plot of some fluorescent response data from flow cytometry investigation of fluid samples with 120 nm beads having fluorescent labels and a second graphical plot of some blank flow cytometry data of clean buffer solution, with data of both plots collected by a first fluorescence detection channel of a flow cytometer.

FIG. 6 illustrates a first graphical plot of some fluorescent response data from flow cytometry investigation of fluid samples with 120 nm beads having fluorescent labels and a second graphical plot of some blank flow cytometry data of clean buffer solution, with data of both plots collected by a second fluorescence detection channel of a flow cytometer.

FIG. 7 illustrates an example of a selected portion of the blank flow cytometry data with data value voltage plotted relative to time, and showing examples of a baseline background signal level of the selected portion and a minimum signal threshold requirement set relative to the baseline background signal level.

FIG. 8 illustrates a plot of peak width (as time duration) vs. peak height (as voltage magnitude) for some example flow cytometry fluorescent response data and illustrating some different possible fluorescent response populations for a stained fluid sample with stained nanoparticles.

FIG. 9 illustrates two example temporal peaks evaluated for the same fluorescent response emission signature for two different nanoparticles stained with the same fluorescent stain but having different particle sizes and having peak properties indicating different particle sizes.

FIG. 10 illustrates an overlay of two time-correlated temporal peaks detected for the same nanoparticle by two different fluorescence detectors detecting separately for different fluorescent emission signatures of two different fluorescent stains.

FIG. 11 illustrates a cargo loading example with a first temporal peak from detection on one detection channel for fluorescence response of a lipophilic membrane dye and several possible examples of a second, coincident temporal peak from detection on another detection channel for fluorescence response of a nucleic acid luminal dye.

FIG. 12 illustrates an example of a computing device configured as a flow cytometry data analysis system including computer capabilities for computer-implementation of a flow cytometry data analysis operation as a computer-implemented method.

FIG. 13 illustrates an example of a flow cytometry system.

### DETAILED DESCRIPTION

An “event” in flow cytometry refers to detection through analysis of flow cytometry response data that a particle, typically a single particle, passed through the investigation zone of a flow cytometer. An objective of analyzing flow cytometry response data is to accurately identify and count all individual particle occurrences in the fluid sample, that is to reduce the number of identified “events” that are in fact false positives (which can lead to overcounting of particles) while also reducing instances of actual particle occurrences not being identified as “events” (which can lead to undercounting of particles). For convenient and clear reference, “events” may be equivalently referred to herein as “detection events” or “particle detection events”. A common approach to identifying “events” is to apply an algorithm to flow cytometry response data to identify peaks in a time series of detected data values having a peak height exceeding a minimum signal threshold requirement, which is typically set at some level above a mean background signal level of the flow cytometer, thus differentiating background signal characteristics from data characteristics that are indicative of actual particle events. Setting a minimum signal threshold requirement too low risks increasing potential for identifying false positives as “events”, leading potentially to significant overcounting of particles, while setting a minimum signal threshold too high risks missing identification of actual particle occurrences as “events”, leading potentially to significant undercounting of particles.

The present disclosure is directed to flow cytometry investigation of fluid samples to identify and count nanoparticles from fluorescent response data. For purposes of the present disclosure “nanoparticles” refers to particles smaller than about 1000 nanometers (smaller than about 1 micron) in maximum cross-dimension, and which nanoparticles are not contained in a larger particle unit not of nanoparticle size, for example not virus or other particles or substructures contained within a cell or other larger particle structure). This is distinguished from more traditional flow cytometry used to analyze fluid samples to identify and count cells and similarly-sized particles that tend to be at a few, and often several, microns in size.

With the techniques of the present disclosure, detection events corresponding to nanoparticles may be determined based solely on fluorescent response data characteristics, and without use of traditional particle identification from light scatter data. With the techniques of the present disclosure, fluorescent response data analysis may be employed as an initial and primary technique for identifying nanoparticle detection events, as distinguished from utilization of fluorescent response data as a secondary screening tool for characterization of particle attributes of particle detection events identified solely or primarily from detected light scatter data.

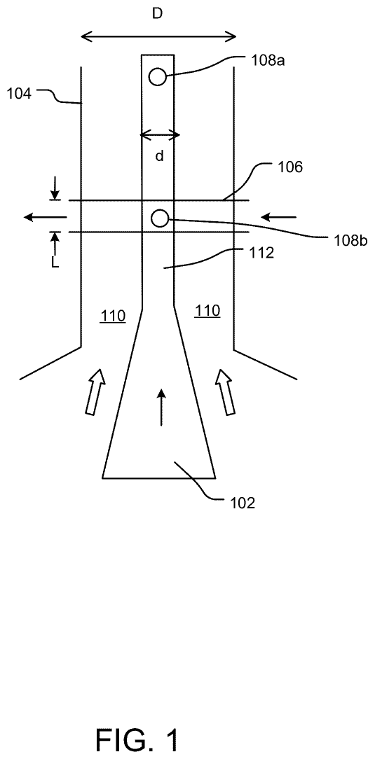

FIG. 1 illustrates a simple example of performing a flow cytometry investigation using a single laser source for excitation radiation in a flow cytometry system in which a stained fluid sample to be investigated is hydrodynamically focused with sheath fluid. As used herein, a “stained fluid sample” means a fluid sample containing material to be investigated by flow cytometry for presence of nanoparticles and which is stained with one or more fluorescent stains configured to stain, within the composition of the fluid sample, one or more features of the nanoparticles of interest for evaluation. Fluorescent stains attached to the nanoparticles provide a fluorescent emission response when subjected to excitation energy, typically radiation, such as light from a laser, LED, or other source. Some examples of fluorescent stains include the so-called fluorogenic dyes that bind non-specifically to particular particle features, for example to membrane proteins or to nucleic acid, but not to a specific binding site. The fluorogenic dyes exhibit relatively low fluorescence when in an unbound state free in solution and exhibit much greater fluorescence when bound to a particle feature. Other examples of fluorescent stains include those that bind to specific sites e.g., epitopes). Some examples of binding site-specific fluorescent stains are fluorescent antibody stains including a fluorescent molecule conjugated to an antibody, which is capable of site-specific binding at a particular epitope, either directly or indirectly by binding to another antibody specific for directly binding to the particular epitope. Unlike the fluorogenic dyes, fluorescent antibody stains tend to be fluorophores that have a high level of fluorescence whether or not bound to a nanoparticle. Other examples of site-specific antibody stains are fluorescent aptamer stains, which are similar to antibodies but with a fluorescent moiety bound to an aptamer instead of an antibody.

As shown in the example of FIG. 1, flow of a stained fluid sample 102 is directed through an investigation channel 104 where the flow of the stained fluid sample 102 is subjected to excitation radiation in an investigation zone 106 within the investigation channel 104, with the investigation zone 106 illustrated as a focal zone of a laser beam illuminating an area within the investigation channel 104. FIG. 1 shows two example particles 108a,b in the flow of the stained fluid sample 102. One particle 108a has already passed by the investigation zone 106 and the other particle 108b is passing through the investigation zone 106. The particles 108a,b are stained with fluorescent stain. When the fluorescent stains are excited by the excitation radiation of the laser beam in the investigation zone 106, the fluorescent stain on the particles 108a,b emits a fluorescent emission that is detected by a detector (e.g., a photomultiplier tube) that provides the flow cytometry fluorescent response data for evaluation to identify the presence of the particle and for particle characterization. The particles 108a,b may be stained with multiple fluorescent stains that provide different fluorescent emission signatures (e.g., that fluoresce at different wavelengths) which are then separately detected and analyzed. The separately detected fluorescent data can be time correlated and each analyzed both for identification of the presence of a particle and for particle characteristics.

In the illustration of FIG. 1, the flow of the stained fluid sample 102 is hydrodynamically focused at the entrance to the investigation channel by a surrounding flow of sheath fluid 110 that transforms the flow of the stained fluid sample 102 to a hydrodynamically-focused flow in a small core stream 112 when subjected to the laser beam in the investigation zone 106.

FIG. 1 also shows some dimensional features, including a cross-dimensional width “D” of the investigation channel, a cross-dimensional width “d” of the core stream 112, and a length “L” of the investigation zone 106 in a direction of sample flow through the investigation channel 104. In this example, the dimensional value L is the width of a laser beam across which the nanoparticles 108 pass for investigation in the core stream 112 within the investigation zone 106. Although the cross-sectional profile of such an investigation channel 104 can be any convenient shape (e.g., circular, semicircular, rectangular), a rectangular cross-sectional shape is common, with a square cross-sectional shape preferred (in which case a depth of the investigation channel would equal the width (D) of the investigation channel). The investigation channel 104 may be, for example, provided in a flow cell or may be provided as a microfluidic feature on a microfluidic chip. As will be appreciated, the size of the core stream (d) will depend upon the ratio of the volumetric flow rate of the flow of the sheath fluid 110 to the volumetric flow rate of the flow of the stained fluid sample 102 through the investigation channel. A higher ratio results in a greater degree of hydrodynamic focusing of the flow of the flow of stained fluid sample 102 and a smaller width of the core stream 112. As will also be appreciated, as the flow cross-section of the investigation channel 104 increases, a higher ratio of volumetric flow rate of the sheath fluid to the stained fluid sample will be required to achieve a core stream of the same with core width (d).

As the flow rate, and therefore the flow velocity, of the flow of the stained fluid sample 102 through the investigation zone 106 (past the laser beam) decreases, the longer the time that the particle 108b will be subjected to the excitation energy of the laser beam in the investigation zone 106, permitting a greater number of fluorescent response readings to be taken by a detector. As will be appreciated, an expected number of fluorescent response data values generated per nanoparticle in the investigation zone 106 is a function of the length L of the investigation zone 106, the velocity of the fluid sample in the core stream 112 passing through the investigation zone 106, and the frequency at which a fluorescent response detector takes a measurement and provides a data value output for detected fluorescent response.

Very low flow rates and flow velocities provide a benefit of a greater number of fluorescent response readings being taken for each particle passing by the laser beam 106, but also lengthen the time required to process stained fluid samples of a given volume, which may become too long for practical use. Also, achieving very low flow velocities in flow channels of typical cross-sectional dimensions can require very high ratios of the volumetric flow rate of the sheath fluid to the volumetric flow rate of the stained fluid sample.

Flow cytometry fluorescent response data includes a time series of fluorescent response data values representative of fluorescent signal intensity as periodically measured and outputted by a radiation (light) detector detecting for the fluorescent signal. The fluorescent response data values accordingly correspond to detected magnitudes of the fluorescent response during a flow cytometry investigation. The number of fluorescent response data values in the time series will depend upon the length of time that a flowing fluid sample is subjected to fluorescent response measurement in an investigation channel during flow cytometry and the frequency at which the detector takes a measurement and provides a data value output. The length of time for collecting the time series of fluorescent response data values will depend upon variables including the sample size and flow rate of the stained fluid sample through the investigation channel. Magnitudes of the data values will vary depending upon gain settings of detectors.

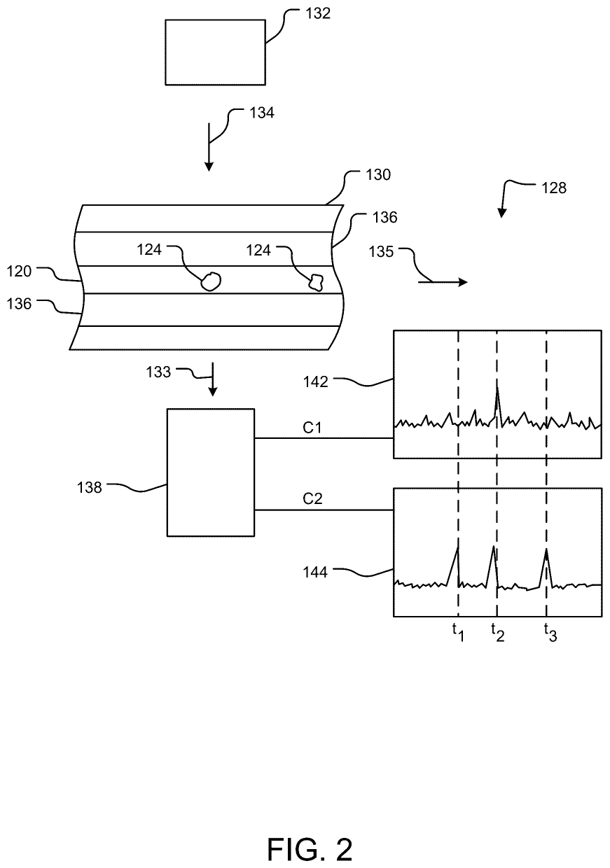

FIG. 2 illustrates an example of partial componentry of a flow cytometer 128 during performance of a flow cytometry investigation of a stained fluid sample 120 stained with two different fluorescent stains each providing a different fluorescent emission signature from stained particles 124. The illustrated componentry of the flow cytometer 128 includes an excitation radiation source 132 (e.g., a laser), an investigation channel 130 and a detection and data output system 138. As shown in FIG. 2, the flowing stained fluid sample 120, and the particles 124 within the stained fluid sample 120, are in a core stream surrounded by flow of a sheath fluid 136 in the investigation channel 130 flowing in a direction indicated by a flow arrow 135. The flowing stained fluid sample 120 is subjected to excitation radiation 134 from the excitation radiation source 132, and a fluorescent response 133 from the investigation channel 130 is detected by the detection and data output system 138. The detection and data output system 138 includes separate radiation detectors for the different fluorescent response signatures of the fluorescent stains in the stained fluid sample 120. Such photodetectors can be for example photomultiplier tubes, silicon photomultipliers, avalanche photodiodes, or selection photodiodes, with photomultiplier tubes being generally preferred.

The detection and data output system outputs electrical signals with a time series of fluorescent response data values corresponding to detected radiation from the different radiation detectors. FIG. 2 shows example outputs of a first time series plot 142 of fluorescent response data values for a first output channel (C1) corresponding to one radiation detector and a second time series plot 144 of fluorescent response it data values for a second output channel (C2) corresponding to the other radiation detector. For example, the time series plots 142 and 144 can be plots of output voltage data values from the respective radiation detectors versus time. As will be appreciated, the magnitudes of the outputted voltage data values may be affected by gain settings of the radiation detectors.

A significant issue with flow cytometry is analysis of the flow cytometry response data to accurately identify events corresponding to detection of stained particles in a stained fluid sample. This analysis includes evaluating the time series of fluorescent response data values to identify temporal peaks indicative of passage of a particle stained with a fluorescent stain through the investigation channel. For illustration purposes, the time plot 142 includes a prominent peak at time t2, indicating a possible event of a particle 124 stained with a first fluorescent stain, and the time plot 144 includes three prominent peaks at times t1, t2 and t3, indicating possible events of three particles 124 stained with a second fluorescent stain. The coincidence of peaks at t2 on both of the time series plots 142, 144 indicates possible passage of a particle stained with both of the different fluorescent stains of the stained fluid sample 120.

Identification of a temporal peak in a time series of fluorescent response data values as corresponding to an event indicative of passage of a particle stained with a fluorescent stain through an investigation channel during a flow cytometry investigation is referred to herein for convenience and clarity brevity as a “detection event”, as noted previously. Identifying a temporal peak as a detection event involves determining that characteristics of the temporal peak satisfy an event condition, in that the characteristics of the temporal peak satisfy one or more requirements that constitute the event condition.

The event condition typically includes at least a minimum signal threshold requirement, and with preferred implementations of the present disclosure the event condition also includes a minimum peak width requirement.

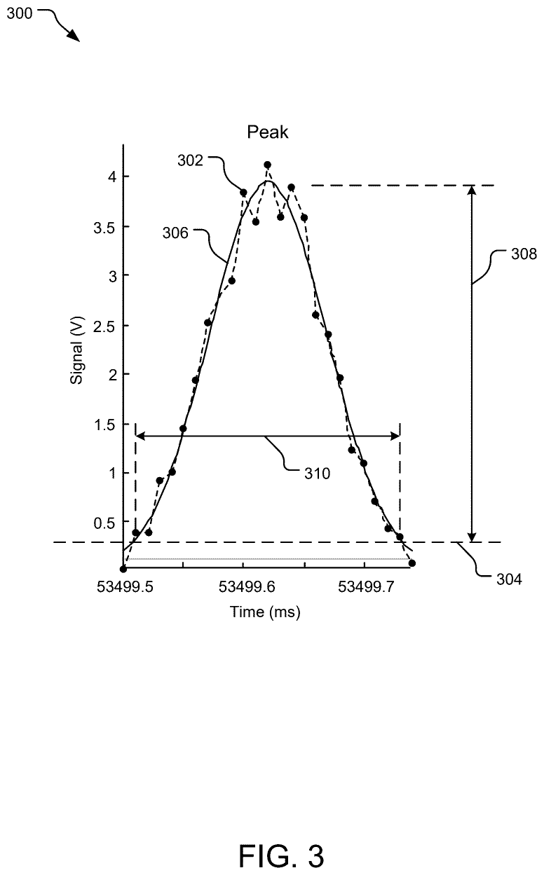

FIG. 3 illustrates an example of a temporal peak 300 on a time series plot of voltage magnitudes of fluorescent response data values 302 (voltage magnitudes) versus time. FIG. 3 shows the raw voltage data values 302 relative to an example minimum signal threshold requirement 304 for voltage value magnitude and a Gaussian curve fit 306 of the fluorescent response data values that exceed the minimum threshold requirement 304. FIG. 3 also shows an example peak height 308 for the temporal peak 300, and an example peak width 310 for the temporal peak 300. The peak height 308 and peak width 310 are characteristics of the temporal peak 300 which may be analyzed for satisfaction of possible requirements of an event condition. As illustrated in FIG. 3, the peak height 308 and the peak width 310 are illustrated relative to the fitted Gaussian curve, which is a preferred convention for evaluating peak height and width with the present disclosure. Alternatively, however, peak height and peak width characteristics could be evaluated based on a peak height represented by a maximum fluorescent response data value 302 and the temporal separation of first and last fluorescent response data values 302 of the temporal peak that exceed the minimum signal threshold requirement 304. Also, peak width represents a temporal span of the peak, but can be identified as a number of the fluorescent response data values 302 that satisfy the minimum signal threshold requirement 304 or an elapsed time that the temporal peak 300 is above the minimum signal threshold requirement 304 (as represented either by the Gaussian curve fit 306 or the temporal positions of the first and last fluorescent response data values that satisfy the minimum signal threshold requirement 304.

In the example of FIG. 3, the temporal peak 300 will satisfy the event condition to qualify as a detection event if the event condition requires only that the temporal peak 300 satisfies the minimum signal threshold requirement 304 in that the peak height 308 exceeds the minimum signal threshold requirement 304. With preferred implementations of the present disclosure, the temporal peak 300 will satisfy the event condition only if the temporal peak also satisfies a minimum peak width requirement. The minimum peak width requirement can be identified as a property of the peak corresponding to a width characteristic of the peak 300. The minimum peak width requirement can be assessed, for example, relative to the peak width 310 (that is relative to the full width of the peak measured at the level of the minimum signal threshold requirement 304). Alternatively, however, a different peak characteristic indicative of peak width can be used, or multiple different peak characteristics indicative of peak width can be used. One example characteristic indicative of peak width is the half-peak width, that is the width of the peak 300 at one-half of the peak height 308 (sometimes referred to as “peak width half max”). Peak width measurements at other fractional magnitudes of the peak height 308 could also be used. Another example characteristic indicative of peak width is a ratio between peak area and peak height. Such a peak width characteristic can be determined from a curve fitted to the peak (e.g., Gaussian curve fit 306), or from analysis directly of the fluorescent response data values 302. One assessment of minimum peak width requirement can be to identify a minimum plurality number of the fluorescent response data values 302 of the peak that must exceed the minimum signal threshold requirement 304.

A minimum signal threshold requirement, and a minimum peak width requirement when part of an event condition, can be set based on an evaluation of blank flow cytometry data from blank sample flow cytometry investigation, which is performed on one or more blank fluid samples not including (being in the absence of) particles stained with the fluorescent stain that will be an object of evaluation using the event condition for flow cytometry investigations directed to identifying the stained nanoparticles. In a preferred implementation, setting a minimum signal threshold requirement is based on analyzing a portion of the blank flow cytometry data that excludes some of the blank flow cytometry data, and more particularly an excluded portion of the blank flow cytometry data comprises some or all of a largest 10 percent (top decile) of the background signal data values of the blank flow cytometry data. When the event condition includes a minimum peak width requirement, in a preferred implementation the minimum peak width requirement can be determined in combination with the minimum signal threshold requirement based on the evaluation of the blank flow cytometry data. When a flow cytometer is configured to detect separate fluorescent emission responses from separate fluorescent stains, for example when a fluid sample is stained with multiple different fluorescent stains, a separate minimum signal threshold requirement, and when applicable a separate minimum peak width requirement, should be set for separate event conditions applicable to each of the different fluorescent stains based on separate sets of blank flow cytometry data collected by the different corresponding detectors for the fluorescent emission signatures of the different fluorescent stains. The blank flow cytometry data from each of the different detectors can be collected, however, as part of the same blank sample flow cytometry investigation. Preferably, a blank sample flow cytometry investigation used to set one or more requirements of an event condition should be performed on the same flow cytometer as will be used for the flow cytometry investigations of stained fluid samples to generate flow cytometry response data that will be evaluated relative to the event condition.

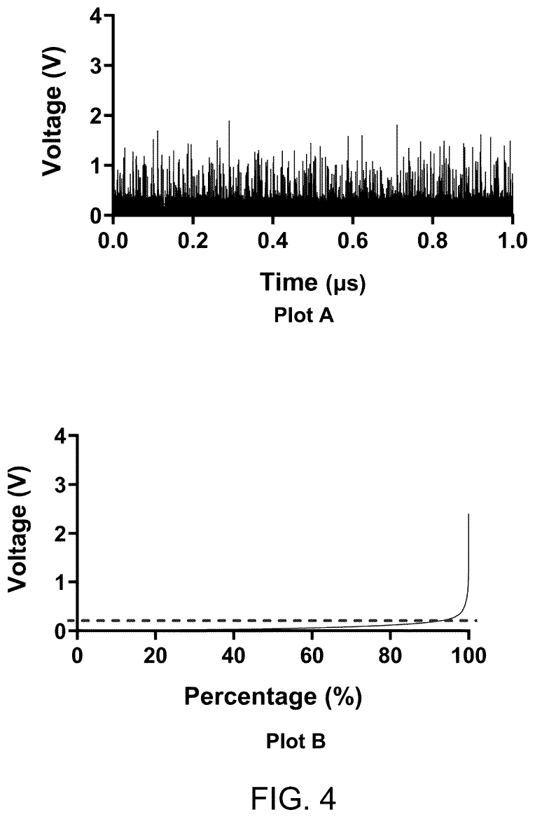

FIG. 4 shows two plots. Plot A is a raw data histogram (time series) of a one second interval of blank flow cytometry data from blank sample flow cytometry investigation of a clean buffered solution (in the absence of material sample with nanoparticles for evaluation) performed on a Virus Counter® 3100 flow cytometer. Plot A shows the collected background signal data values (voltage magnitudes) vs. time collected by one of the fluorescent response detection channels of the flow cytometer. Plot B is a plot of the raw time series data that has been sorted by data value magnitude (voltage magnitude) and with data value magnitude plotted from smallest to largest and with a curve fitted to the plotted data value magnitudes, and with the x-axis identifying the cumulative percentage of data values. A large proportion of the voltage data values (over 95%) are very small, but there is a sharp upward bend in the curve at around 95%, and with only about 5% or fewer of the voltage data values being anomalously large. For convenience, the portion of the data values with larger magnitudes starting at the sharp upward bend may be referred to as the “anomalous portion” of the blank sample data values.

The inventors have identified that this effect can be beneficially employed by setting one or more requirements of an event condition, including a minimum signal threshold requirement and a minimum peak width requirement, by using a portion of the background signal data values that does not include another portion of the background signal data values with the largest data value magnitudes. The portion of the background signal data values excluded from use to set the minimum signal threshold value is referred to herein as the “excluded portion” of the background signal data values. It is preferred that the excluded portion will include most, and even more preferably all, of the anomalous portion of the blank sample data values, that is the blank sample data values with the largest magnitudes starting with those blank sample data values at or possibly just before the beginning of the sharp upward bend of the cumulate magnitude distribution profile, such as that illustrated in Plot B of FIG. 4. The portion of the blank flow cytometry data not including the excluded portion of the background signal data values is sometimes referred to herein for convenience as the “selected portion” of the blank flow cytometry data, that is, the portion containing the background signal data values other than the excluded portion of the background signal data values.

Also, although the data value magnitude distribution profiles of different formulated blank fluid sample compositions will differ somewhat, both in terms of the sharpness of the upward bend and the proportion of the background signal data values that are anomalously large, the magnitude distribution profiles nevertheless tend to follow the same general pattern with a large majority of background signal data values at a low level and a sharp upward bend in the cumulative distribution curve with a much smaller number of the much larger background signal data values. Even stained fluid samples with stained nanoparticles for detection tend to show a similar data value magnitude distribution profile with a large majority of the data value magnitudes being very small. Some different possible compositions for a blank fluid sample that is subjected to blank sample flow cytometry investigation could be, for example, a clean buffer-only solution (such as the clean buffered solution of FIG. 4), a reagent-only fluid sample (such as an aqueous buffered solution with added reagents, such as fluorescent stains, but no nanoparticles or sample material for evaluation), or some other representative composition not including stained nanoparticles that are to be the subject of subsequent flow cytometry investigations using the event condition that is set using the blank flow cytometry data. Also, because the magnitude distribution profiles between different compositions tend to be similar, differing mostly in the size of the small percentage of anomalously large magnitudes, it is generally preferred to use a buffer-only blank fluid sample for performing the blank sample flow cytometry investigation for setting one or more requirements of the event condition.

Data value magnitude distribution profiles of background signal data values from a blank sample flow cytometry investigation will also vary between different flow cytometer designs, for example as a consequence of differences in flow cytometer limits of detection, sample flow rates, and measurement sampling frequency. Accordingly, the profile of the background signal data values sorted by magnitude value may vary between flow cytometers, for example the sharpness of the upward bend of the anomalous portion or the number or percentage of background signal data values in the anomalous portion may vary, but a large majority of the background signal data values should ordinarily be outside of the anomalous portion and the techniques for analyzing and setting event condition requirements by excluding a portion of background signal data values with the largest magnitudes, and preferably most or all of the anomalous portion, should be applicable across different flow cytometer designs although an optimal size for the excluded portion may vary significantly between different particular background signal data value magnitude distribution profiles of different flow cytometer designs. For example, for some flow cytometer designs optimal operation may result in removal of only a top few percent of the background signal data values while for some other flow cytometer designs optimal operation may result in removal of more than all of the top 10% (top decile) of the background signal data values, although as will be appreciated in each case at least some of the top 10% (top decile) of the background signal data values will ordinarily be removed. Also, even if the excluded portion is not optimized for a particular flow cytometer design, still assessing background signal characteristics with exclusion of at least some of the top 10% (top decile), and preferably at least the background signal data values with the largest magnitudes, will improve the setting of event condition requirements, such as a minimum signal threshold requirement, even if not fully optimized for the particular flow cytometer, and especially when such a technique is combined with setting an event condition to include both a minimum signal threshold requirement and a minimum signal width requirement as disclosed herein.

In a preferred implementation, a background signal level is determined for the selected portion of the blank flow cytometry data and the minimum signal threshold is then set at a level higher than that background signal level, and preferably at a level of the average of the background signal data values of the selected portion plus an added amount equal to one or more times a standard deviation value of the background signal data values of the selected portion. In some preferred implementations, the added amount will be between 1.0 and 4.0 times the standard deviation value, more preferably between 1.5 and 3.0 times the standard deviation value, and even more preferably often between 1.8 and 2.5 times the standard deviation value.

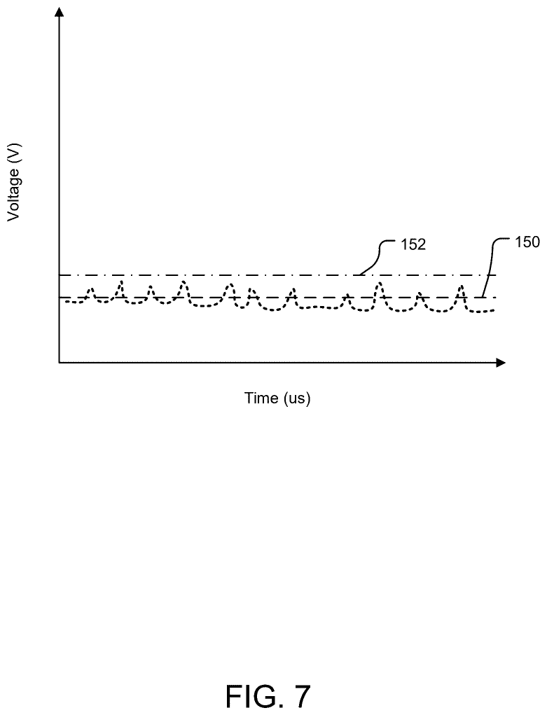

FIG. 7 illustrates an example of a selected portion of the blank flow cytometry data with data value voltage plotted relative to time, and showing an example of a baseline background signal level 150 of the selected portion and an example of a minimum signal threshold requirement 152 set relative to the baseline background signal level 150.

A minimum peak width requirement of the evaluation condition can also be set using the blank flow cytometry data, in combination with setting a minimum signal threshold requirement. This can be accomplished by selecting a combination of the minimum signal threshold requirement and the minimum peak width requirement that when applied to the blank flow cytometry data (all of the blank flow cytometry data, including the selected portion and the excluded portion) results in only a minimal number of temporal peaks in the blank flow cytometry data being identified as detection events satisfying the event condition (false positives). Although that number should be small, it should preferably not be zero and rather should be a small positive number, as zero false positives indicates that the event condition might not be sensitive enough and could lead to undesirable undercounting of stained particles during flow cytometry investigation of stained fluid samples.

The minimum peak width requirement can conveniently be set at a minimum plurality number of fluorescent response data values (preferably a minimum plurality number of consecutive ones of fluorescent response data values) that each satisfy the minimum signal threshold requirement. In setting the minimum peak width requirement, consideration can also be given to the particulars of the flow cytometer that will be generating the fluorescent response data that will be evaluated relative to the event condition. For example, a flow cytometer with design and operating characteristics expected to lead to a larger number of fluorescence measurements per particle passing the excitation radiation (e.g., as a consequence of relatively low sample flow rate and/or relatively high measurement frequency) might be set to require a larger number of fluorescent response data values for the minimum peak width requirement than a flow cytometer with design and operating characteristics expected to lead to a smaller number of fluorescence measurements per particle passing the excitation radiation (e.g., as a consequence of relatively high sample flow rate and/or relatively low measurement frequency). As will be appreciated, the lower the volumetric flow rate of the stained fluid sample and the higher the measurement frequency, the higher will be the number of expected data values obtained per particle.

Once the requirement(s) are set for a detection condition, the event condition can be used for evaluation of fluorescent response data from flow cytometry investigations of stained fluid samples performed over a period of time, but occasionally one or more of the requirements should be reevaluated and reset for enhanced accuracy of flow cytometry evaluation over time. It is preferred that fluorescent response data from flow cytometry investigations of stained fluid samples be performed using detection condition requirements set from blank flow cytometry data from a blank sample flow cytometry investigation that is relatively contemporaneous with the flow cytometry investigations of the stained fluid samples for which the event condition is used for evaluation. In preferred implementations, a blank sample flow cytometry investigation will be performed and resetting of one or more of the requirements of the event condition will occur at least on every day that the flow cytometer is used for flow cytometry investigations of stained fluid samples to be evaluated using the event condition. However, it has been found that when an event condition includes both a minimum signal threshold requirement and a minimum peak width requirement, that the minimum signal threshold requirement should typically be reevaluated and reset with greater frequency than the minimum peak width requirement. The minimum peak width requirement tends to be more associated with the flow cytometer design and instrumentation and may not need to be reset with the same frequency as the minimum signal threshold requirement. In one implementation, reevaluation and resetting of the minimum peak width requirement for a flow cytometer may require a maintenance service visit, whereas resetting of the minimum signal threshold requirement may routinely be performed by flow cytometer users as part of normal flow cytometry procedures. Alternatively, users can be permitted to reset the minimum peak width requirement. Resetting of any one or more, or all of, requirements of an event condition based on updated blank flow cytometry data can be performed manually or can the automated with a computer-implemented evaluation and/or resetting procedure. Such computer implementation can be performed locally to the flow cytometer or can be performed remotely to the flow cytometer. A computer device used to analyze blank flow cytometry data and to reset one or more requirements of an event condition can be part of on-board componentry of the flow cytometer, may be separate from the flow cytometer but locally located relative to the flow cytometer or may be remotely located relative to the flow cytometer. Such a computer device can be communicatively connected to the flow cytometer during some or all of analysis of blank flow cytometry data and/or setting of one or more requirements, for example to access blank flow cytometry data store in computer storage of the flow cytometer, or may be not communicatively connected with the flow cytometer, for example when the computer device contains or has other access to computer readable media having stored therein the blank flow cytometry data.

In addition to use in setting requirements for an event condition, blank flow cytometry data from a blank sample flow cytometry investigation can also be advantageously used for quality control purposes. Such quality control usage can be in connection with setting one or more requirements of an event condition (e.g., setting a minimum signal threshold requirement) or as desired to check performance of the flow cytometer. For either use, the blank flow cytometry data can be evaluated for one or more indications of a possible performance problem with the flow cytometer, and when a possible performance problem is identified then a background data warning notification can be issued, for example advising that the flow cytometer should be subjected to a cleaning operation, subjected to other maintenance, or checked for presence of possible conditions that could negatively affect flow cytometry performance. For example, deficient performance of a flow cytometer can be caused by prior sample carryover, dirty setup (e.g., dirty flow cytometer flow componentry or dirty or past-expiration sample preparation reagents), poor sample preparation protocol (e.g., introducing contaminants), optical componentry misalignment, air bubbles in the fluid conduction path, or other device malfunction issues. In response to such a background data warning notification, the flow cytometer user can take corrective action, such as to run a cleaning cycle, purge fluid lines, or perform or schedule other maintenance on the flow cytometer or operating practices (e.g., to assess and correct as needed optical componentry misalignment, regarding reagent sources or storage and handling protocols, or regarding sample preparation protocols).

In connection with evaluating blank flow cytometry data either with an objective of setting one or more requirements for an event condition or with an objective to simply evaluate flow cytometer performance for possible operational problems, when evaluation of blank flow cytometry data indicates a possible problem with operation of the flow cytometer, corrective action can be taken (e.g., performance of a cleaning cycle or any other corrective action or actions noted in the prior paragraph), and after completing the corrective action or actions, new blank flow cytometry data can be collected on one or more newly prepared blank fluid samples for a new blank sample flow cytometry investigation and the new blank flow cytometry data can again be evaluated for quality control purposes, and if the new blank flow cytometry data evaluation does not indicate possible flow cytometer performance problems (e.g., as a consequence of the corrective action(s)), then normal use of the flow cytometer to perform flow cytometry investigations of stained fluid samples can continue with greater confidence in flow cytometer performance and/or the new blank flow cytometry data can be used to set (or reset) one or more requirement of an event condition.

As one example of use of blank flow cytometry data for quality control purposes, the blank flow cytometry data can be evaluated relative to the then-current evaluation condition requirement setting(s) or proposed setting(s) (e.g., minimum threshold requirement and minimum peak width requirement), and if the blank flow cytometry data evaluated relative to such setting(s) identifies more than a preset threshold of detection events (false positives) in the blank flow cytometry data, then a background data warning notification can be issued. Such a preset threshold can be set based on characteristics and experience with the particular flow cytometer design, for example as a multiple (e.g., at least 1.5, or at least 2) times an expected number of false positive detection events for blank flow cytometry data from the flow cytometer. Such an expected number of detection events (false positives) can be, for example, a number of false positive detection events used as a background condition for setting the minimum signal threshold requirement and the minimum peak width requirement of the event condition used to evaluate the blank flow cytometry data for quality control purposes.

As will be appreciated, raw fluorescent response data from a flow cytometry investigation of a stained fluid sample needs to be acquired and stored (either locally or remotely, but preferably locally) contemporaneously with performance of the flow cytometry investigation as the raw data is generated. Evaluation of the fluorescent response data relative to an event condition can be performed contemporaneously with collection and logging of raw fluorescent response data or can be performed at a later time using the stored fluorescent response data. As with analysis of blank flow cytometry data, analysis of fluorescent response data from flow cytometry investigation of a stained fluid sample can occur locally or remotely relative to the location of the flow cytometer.

Temporal peaks in flow cytometry fluorescent response data can also be evaluated relative to other criteria in addition to a minimum signal threshold requirement and a minimum peak width requirement, and such other criteria can be additional requirements of an event condition for identifying a detection event or, preferably, can be applied as secondary screening criteria for further analyzing detection events, for example for the occurrence in the fluorescent response data of incidences of swarm, (e.g., particle coincidence, with two particles within the influence of the excitation radiation in the investigation channel at the same time and generating overlapping fluorescent responses or particle aggregations, with several particles in the form of an aggregate passing through the investigation channel together).

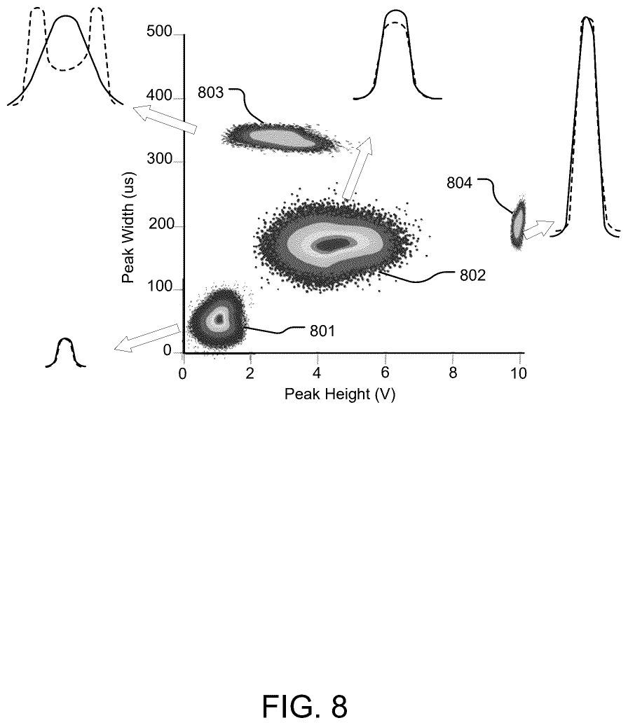

FIG. 8 shows a plot of peak width (as a function of time) vs. peak height (voltage magnitude) for some example flow cytometry fluorescent response data and illustrating some different possible fluorescent response populations for a stained fluid sample with stained nanoparticles. Also illustrated are some representative temporal peak profiles for such different populations, and also showing representative Gaussian fits to such temporal peaks. In this example, the fluorescent response data includes four example populations of fluorescent response. A first population 801 of fluorescent response data, having small peak height and peak width, is indicative of impurities, and which should largely be avoided as detection events by requirements of the event condition to identify nanoparticle detection events, for example by an event condition including a minimum signal threshold requirement and minimum peak width requirement as described herein. A second population 802 of fluorescent response data, having intermediate peak width and intermediate peak height, is indicative of single-particle nanoparticles. Particle populations can also be identified as associated with particle swarm. Particle swarm is a condition where more than one particle (here the nanoparticles of interest for evaluation) is present within the focal area of the excitation radiation (e.g., laser) at the same time. Some examples of particle swarm include particle coincidence and particle aggregation. A third population 803 of fluorescent response data shown in FIG. 8, having large peak width and intermediate peak height, is indicative of occurrence of particle swarm in the form of particle coincidence. The representative temporal peak shows two peaks separated by an intermediate valley that does not return to a background baseline, indicating overlapping signals of two particles passing through the investigation channel at the same time, but when converted to a Gaussian fit appear as a single peak, and will have a very low degree of fit to the actual data points. A fourth population 804 of fluorescent response data, having a very large peak height, is indicative of occurrences of particle swarm in the form of particle aggregation, where occurrences of particle aggregations passing through the investigation channel provide temporal peak profiles appearing to be that of a larger single particles, as opposed to the nanoparticles that are the object of the evaluation. Evaluating fluorescent response data for temporal peak attributes characteristic of particle swarm (e.g., as particle coincidence or particle aggregations) can provide important information on flow cytometry performance and possible performance problems. For example, a high identified incidence of particle coincidence can be indicative of the sample subjected to flow cytometry having a larger concentration of particles than is optimal for accurate flow cytometry quantification in that flow cytometer, whereas a high identified incidence of particle aggregations can be indicative of a problem associated with fluid sample chemistry or preparation or handling protocols that lead to the presence of particle aggregates in the stained fluid sample.

Screening temporal peaks, for example the temporal peaks of detection events, by supplemental criteria through secondary screening of detection events can be used for example to fine-tune qualification of temporal peaks to be counted as particle occurrences and/or to issue data warning notifications, for example when a permissible threshold number of detections indicative of particle swarm (e.g., particle coincidence events and/or aggregated particle events) have been identified.

For example, temporal peaks (either all identified temporal peaks with initial evaluated to identify detection events or, preferably, only those temporal peaks already identified as detection events) can be evaluated for identification as qualifying peaks that include one or more peak properties that satisfy a peak qualification condition, which peak qualification condition can be initially applied as a requirement of the evaluation condition or, preferably, can be applied as a secondary screen of already-identified detection events.

As one example, such a peak qualification condition can include a maximum peak width requirement for the peak width of the temporal peak. Such a maximum peak width requirement will be larger than the minimum peak width requirement for a detection event, and such maximum peak width requirement, although temporal in nature, could be expressed as either a maximum permitted time duration of the peak width or a maximum permitted number of sequential data values of the temporal peak. Such a maximum peak width requirement could be set, for example, as some factor of the minimum peak width requirement or as a factor of an expected number of fluorescent data values for each nanoparticle based on operating characteristics of the flow cytometer. Such a maximum peak width requirement could be used to identify potentially problematic temporal peaks, such as those indicative of a particle swarm condition in the form of particle coincidence. As an alternative or a supplement to use of a maximum peak width requirement, a minimum degree of fit requirement could be used to identify potential particle coincidence events. If a degree of fit of a Gaussian curve is lower than a required minimum degree of fit, then the temporal peak can be identified as problematic. When an event of a temporal peak having a peak width exceeding the maximum peak width requirement and/or a degree of fit not satisfying a minimum degree of fit requirement, for example indicative of a particle coincidence event, one action could be to exclude the event from a count of particles. Another possible action could be to count the event as multiple particles, for example to count such an event is two particles in recognition of a coincident particle event. Also, a count can be made of the cumulative number of such events occurring, and when the cumulative number cumulative number of such events exceeds a preidentified permissible threshold measure, a data warning notification can be issued, for example is a warning stored with flow cytometry data and/or as a contemporaneous warning issued on a graphic interface.

As another example, such a peak qualification condition can include a maximum peak height requirement. Such a maximum peak height requirement could be used to identify potentially problematic temporal peaks, such as those indicative of an occurrence of particle swarm in the form of particle aggregations passing through the investigation channel. Preferably, such a maximum peak height requirement would be relative to the minimum signal threshold requirement, and could be set, for example, at some multiple of the minimum signal threshold requirement. When an event of a temporal peak having a peak height exceeding the maximum peak height requirement, for example indicative of a particle aggregation event, one action could be to exclude the event from a count of particles. Also, a count can be made of the cumulative number of such events occurring, and when the cumulative number of such events exceeds a preidentified permissible threshold measure, a data warning notification can be issued, for example is a warning stored with flow cytometry data and/or as a contemporaneous warning issued on a graphic interface.

Detection events of fluorescent response data can also be evaluated for particular particle characteristics.

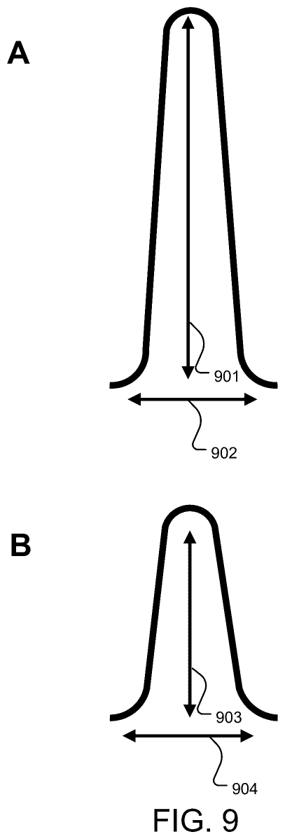

One example is distinguishing between types of nanoparticles when the stained fluid sample includes mixed populations of different nanoparticles. Based on one or more peak properties of the detection events, a particle type can be identified for each of the detection events, or for each qualifying peak when the detection events are subjected to secondary screening such as relative to a maximum peak width requirement and/or a maximum peak height requirement to exclude some temporal peaks as qualifying peaks. Different portions of the evaluated temporal peaks can be identified as belonging to different populations of different types of stained nanoparticles. One example is for nanoparticle populations expected to, or suspected of having, different populations of differently-sized nanoparticles. Discriminating criteria could be, for example, a peak property or properties indicative of particle size, for example indicative of different fluorescent intensities of fluorescent emission responses from different particles the particle. FIG. 9 illustrates two example temporal peaks (A,B) evaluated for the same fluorescent response emission signature (e.g. detections at different times by the same fluorescence detector) for two different nanoparticles stained with the same fluorescent stain but having different particle sizes and having peak properties indicating the different particle sizes. Temporal peak A has a peak height 901 and a peak width 902 and temporal peak B as a peak height 903 and a peak width 904. For example, the stained fluid sample could include a lipophilic fluorescent stain, with affinity to stain a lipophilic feature of a particle membrane, that will provide a higher intensity fluorescence on larger membrane-containing biological particles than smaller membrane-containing biological particles, because of a greater quantity of fluorescent stain being able to bind to the larger membrane of the larger particle than to the smaller membrane of the smaller particle. Temporal peak A corresponds to a larger nanoparticle than temporal peak B, having a significantly larger peak height (and therefore also a much larger peak area indicative of a higher intensity fluorescent response), than temporal peak B, which corresponds to a smaller-sized nanoparticle. Discriminating criteria could include, for example peak height, a combination of peak height and peak width, and/or peak area (the area of the peak above the minimum signal threshold requirement). The discriminating criteria can be correlated to particle size and particles can be identified as belonging to differently sized particle populations, and particles identified with each particle-sized population can be separately counted and reported.

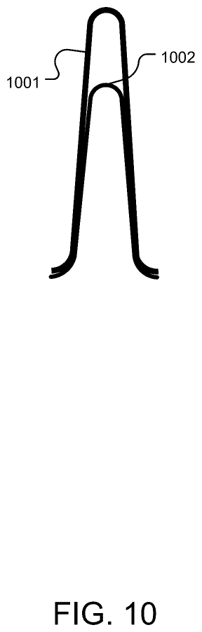

Another example is the use of multiple different fluorescent stains to identify different characteristics of a nanoparticle. FIG. 10 shows an overlay of two time-correlated temporal peaks 1001 and 1002 detected for the same nanoparticle by two different fluorescence detectors detecting separately for different fluorescent emission signatures of two different fluorescent stains. The temporal peaks 1001, 1002 are associated with different characteristics of a nanoparticle because of different staining affinity of the different fluorescent stains for different features of the nanoparticle. As one example, temporal peak 1001 could be detected fluorescence from a lipophilic dye with affinity for staining a biological particle membrane feature and temporal peak 1002 could be detected fluorescence from a luminal dye with affinity for staining a feature of the particle lumen. A “luminal dye” can alternatively be referred to as a “lumenal dye”. For example, the lipophilic dye could be a dye that stains membrane proteins and the luminal dye could be a dye that stains nucleic acid in a particle lumen.

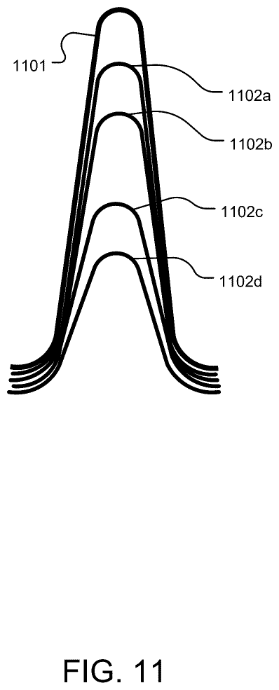

As a variation of this example, a luminal dye could be directed to staining an internal cargo of the nanoparticle, for example a nucleic acid cargo disposed inside of a biological nanoparticle, for example loaded into a lipid-based nanoparticle (LPN) or other pharmaceutical carrier nanoparticle. Peak properties of a luminal stain temporal peak can be evaluated to estimate an extent of cargo loading of the biological nanoparticle with the nucleic acid cargo in the particle lumen, for example to provide a quantitative or semi-quantitative indication of the amount of the nucleic acid cargo in the biological particle. FIG. 11 shows a cargo loading example with a first temporal peak 1101 from detection on one detection channel for fluorescence response of a lipophilic membrane dye and several possible examples of a second, coincident temporal peak 1102a-d from detection on another detection channel for fluorescence response of a nucleic acid luminal dye. The different relative areas of the different examples for the second temporal peak 1102a-d indicate different levels of nucleic acid cargo content in the nanoparticle, with a smaller peak area (e.g., 1102d) correlating to a smaller amount of nucleic acid cargo and a larger peak area (e.g., 1102a) correlating to a larger amount of nucleic acid cardo. Correlation of cargo loading may be based on absolute intensities of the second temporal peaks 1102a-d and/or on the relative intensities between the first temporal peak 1101 and the second temporal peak 1102a-d.

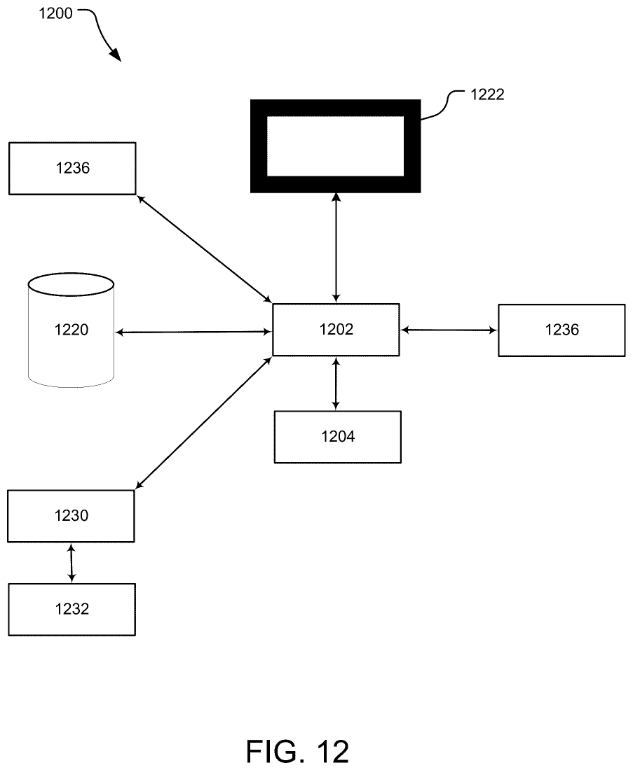

Various methods, or portions of such methods, of the present disclosure can be computer-implemented. For example any one or more, or all of, analyzing blank flow cytometry data (or a portion thereof); determining flow cytometry background signal characteristics; setting a minimum signal threshold requirement based on characteristics of a portion of blank flow cytometry data; setting an event condition (or one or more requirements of an event condition, such as a minimum signal threshold requirement and/or a minimum peak width requirement); and analyzing fluorescent response data can be computer implemented. FIG. 12 illustrates an example of a computing device 1200 configured as a flow cytometry data analysis system including computer capabilities for computer-implementation of a flow cytometry data analysis operation as a computer-implemented method, or portion thereof, of the present disclosure.

The computing device 1200 may be a client computing device (such as a laptop computer, a desktop computer, or a tablet computer), a server/cloud computing device, and Internet-of-Things (IoT), any other computing device, or a combination of these options. The computing device 1200 includes at least one hardware processor 1202. The at least one hardware processor 1202 can include one or more than one hardware processor. The computing device 1200 includes at least one memory 1204 communicatively connected with the at least one processor 1202. The at least one memory 1204 generally includes both volatile memory (e.g., RAM) and non-volatile memory (e.g., flash memory), although one or the other type of memory may be omitted. Volatile and nonvolatile memory are also referred to herein by the alternative terms transitory and non-transitory memory.

An operating system resides in the at least one memory 1204 and is executed by the at least one processor 1202. Optionally, the computing device 1200 includes storage 1220. In the example computing device 1200, one or more programs (e.g., software programs or firmware programs) and/or segments, or other program code in modules are loaded into the operating system on the at least one memory 1204 and/or the storage 1220, and including instructions that when executed by the at least one processor 1202 configure the at least one processor to perform a computer-implemented method, or portion thereof, of the present disclosure.

Optionally, the computing device 1200 can include one or more communication transceivers 1230, which optionally may be connected to one or more antenna(s) 1232 to provide network conductivity (e.g., mobile phone network, Wi-Fi®, Bluetooth®) to one or more other servers, client devices, IoT devices, and other computing and communication devices.

Optionally, the computing device 1200 can include a communications interface 1236 (such as a network adapter or an input/output (I/O) port, which are types of communication devices). The computing device 1200 can use an adapter and any other types of communication devices for establishing connections over a wide-area network (WAN) or local-area network (LAN). It should be appreciated that the network connections shown are exemplary and that other communications devices and means for establishing a communications link can be used.

Optionally, the computing device 1200 can include one or more input devices 1234 such that a user may enter commands and information (e.g., a keyboard, trackpad, or mouse). These and other input devices may be coupled to the server by one or more interfaces, such as a serial port interface, parallel port, or universal serial bus (USB). The computing device 1200 can, optionally, include a display 1222, such as a computer screen, which may have touchscreen capability.

Optionally, the computing device 1200 can include computer memory in a variety of tangible computer-readable (i.e., computer processor-readable) storage media and intangible processor-renewable communication signals. Tangible computer-readable storage media can be embedded in any available computer-readable media that can be accessed, directly or indirectly, by the processor 1202 and can include both volatile and non-volatile storage media. Tangible computer-readable storage media excludes intangible and transitory communication signals (such as signals per se) and includes volatile and nonvolatile, removable and non-removable storage media implemented in any method processor technology for storage of information such as computer-readable instructions, data structures, program modules, and other data. Tangible computer-readable storage media includes but is not limited to RAM, ROM, EEPROM, flash memory or other computer memory technology, CDROM, digital versatile disks (DVD) or other optical disk storage, magnetic cassettes, magnetic tape, magnetic disk storage, or other magnetic storage devices, or any other tangible medium which can be used to store the desired information and which can be accessed, directly or indirectly, by the at least one processor 1202. In contrast to tangible computer-readable storage media, intangible processor-readable communication signals may embody computer-readable instructions, data structures program modules, or other data resident in a modulated data signal, such as a carrier wave or other signal transport mechanism. The term “a modulated data signal” means a signal that has one or more of its characteristics set or changed in such a manner as to encode information in the signal. By way of example, not limitation, intangible communication signals include signals traveling through wired media such as a wired network or direct-wired connection, and wireless media such as acoustic, RF, infrared, and other wireless media.

It should be appreciated that one or more, or all, of the components of the computer system 1200 as illustrated in FIG. 12 can be located locally to one or more, or all, of the other components, and one or more, or all, of the components can be located remotely to one or more, or all, of other of the other components. Communication connections as illustrated in FIG. 12 can be established in any suitable manner with any suitable communication componentry.

The present disclosure also includes articles of manufacture, which excludes software per se. An article of manufacture may comprise a tangible computer-readable media to store logic and/or data. Examples of such computer-readable media include one or more types of computer-readable storage media capable of storing electronic data, including volatile memory or nonvolatile memory, removable or non-removable memory, erasable or non-erasable memory, writable or re-writable memory, and so forth. Examples of the logic may include various software elements such as software components, programs, applications, computer programs, application programs, system programs, machine programs, operating system hardware, middleware, firmware, software modules, routines, subroutines, operation segments, methods, procedures, software interfaces, application program interfaces (API), instruction sets, computing code, computer code, code segments, computer code segments, words, values, symbols, or any combination thereof. In some implementations, for example, an article of manufacture may store executable instructions (computer program instructions) that when executed by at least one processor, cause the processor to perform methods and/or operations in accordance with described embodiments. The executable computer program instructions may include any suitable type of code, such as source code, compiled code, interpreted code, executable code, static code, dynamic code, and the like. The executable computer program instructions may be implemented according to a predefined computer language, manner, or syntax, for instructing at least one processor to perform a certain operation segment. The instructions may be implemented using any suitable high-level, low-level, object-oriented, visual, compiled, and/or interpreted programming language.

The computer-implementations described herein are implemented as logical steps in one or more computer systems. The logical operations may be implemented (1) as a sequence of computer-implemented steps executing in one or more computer systems and (2) as interconnected machine or circuit modules within one or more computer systems. The implementation is a matter of choice, dependent on the performance requirements of the computer system being utilized. Accordingly, the logical operations making up the computer implementations described herein are referred to variously as operations, steps, objects or modules. Furthermore, it should be understood that logical operations may be performed in any order, unless explicitly claimed otherwise or a specific order is inherently necessitated.

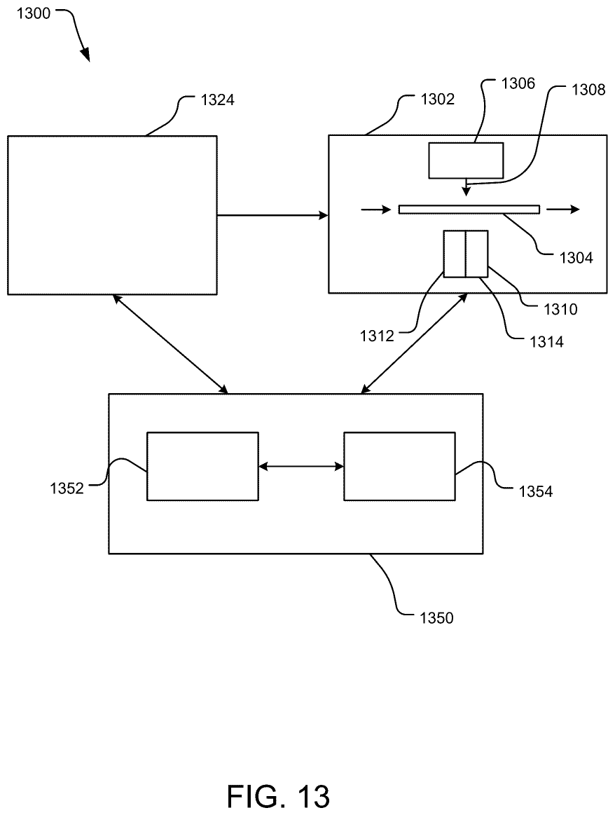

FIG. 13 illustrates an example of a flow cytometry system 1300. The flow cytometry system 1300 includes a flow cytometry investigation system 1302 where fluid samples (e.g., stained fluid samples or blank fluid samples) are subjected to flow cytometry investigation. The flow cytometry investigation system 1302 includes an investigation channel 1304 that provides a controlled flow conduction path for flow of fluid samples for investigation, radiation source 1306 (e.g., a laser) to provide excitation radiation 1308 to the investigation channel 1304 for investigation of the fluid sample, and a radiation detection system 1310 including two detection channels 1312 and 1314 with detectors to detect two different fluorescent emission signatures from the investigation channel 1304, such as could be provided by two different fluorescent stains in a stained fluid sample.

Optionally, the flow cytometry system 1300 also includes an autosampler 1324 configured to automatically deliver fluid samples to the flow cytometry investigation system 1302 for flow cytometry investigation in the flow cytometry investigation system 1302. For example, the autosampler can be configured to automatically deliver to the flow cytometry investigation system 1302 a sequence of a plurality of fluid samples contained in different compartments of a multi-well plate.

The flow cytometry system 1300 includes a data evaluation and control system 1350 communicatively connected to the flow cytometry investigation system 1302, and optionally also the autosampler 1324 when the flow cytometry system 1300 includes the autosampler 1324. The data evaluation and control system 1350 includes at least one processor 1352 and computer memory (preferably including non-volatile media for storing information) 1354 communicatively connected with the at least one processor 1352. The computer memory 1354 has stored therein instructions (e.g., of a computer program) executable by the at least one processor 1352 to direct operation of the flow cytometry investigation system 1302 and the autosampler 1324 to perform flow cytometry investigations of fluid samples. The data evaluation and control system 1350 has controller capabilities, at least to an extent that the data evaluation and control system 1350 is configured to control operation of the flow cytometry investigation system 1302, and optionally to also control the autosampler 1324 when the data evaluation and control system 1350 includes the autosampler 1324, to perform flow cytometry investigations of fluid samples. The data evaluation and control system 1350 also includes data evaluation capabilities, at least to an extent that the data evaluation and control system 1350 is configured to perform as computer-implemented methods, or portions thereof, one or more flow cytometry data analysis operations, for example analyses, evaluations, and/or manipulations of flow cytometry response data (e.g., fluorescent response data and/or blank flow cytometry data).

The data evaluation and control system 1350 receives flow cytometry response data (e.g., blank flow cytometry data from blank sample flow cytometry investigation of blank fluid samples, fluorescent response data from flow cytometry investigation of stained fluid samples) from the radiation detection system 1310 for storage in the computer memory 1354. The computer memory 1354 also has stored therein instructions (e.g., of one or more computer programs) executable by the at least one processor 1352 for analyzing, evaluating, and/or manipulating flow cytometry response data. In preferred implementation, the stored instructions include instruction for adjusting settings for data evaluation (e.g., setting an evaluation condition or setting one or more requirements of an evaluation condition). The stored instructions, when executed by the at least one processor 1352, can include performance of any of the computer-implemented methods, or portions thereof, of the present disclosure. The data evaluation and control system 1350 may include one or more features of the computing device 1200 of FIG. 12. For example, the at least one processor 1352 can be or include the at least one processor 1202 and the at least one computer memory 1354 can be or include the at least one memory 1204 and/or the storage 1220 of the computing device 1200 illustrated in FIG. 12, and with any or all of the attributes discussed in relation to FIG. 12. The at least one computer memory 1354 can be or include one or more tangible processor-readable storage media and intangible processor-renewable communication signals as discussed in connection with FIG. 12.

Components of the data evaluation and control system 1350 include hardware components, including some or all of the at least one computer memory 1354 when in the form of tangible computer-readable media and including the at least one processor 1352 and software components, such as computer program instructions stored in the at least one computer memory or being executed by the at least one computer processor 1352. Some or all of these components of the data evaluation and control system 1350 can be contained in a common enclosure (e.g., flow cytometer housing) with the flow cytometry investigation system 1302 and some or all components of the data evaluation and control system 1350 can be outside of such an enclosure (e.g., a communicatively connected computing device such as a laptop computer, desktop computer, tablet computer and/or computer server). Such components outside of such an enclosure can be located locally (e.g., in the same facility) with the flow cytometry investigation system 1302 or can be located remotely to the flow cytometry investigation system 1302 (e.g., a remote server). Consequently, one or more computer memory of the at least one computer memory 1354 and/or one or more computer processor of the at least one processor 1352 can be located in a common enclosure with the flow cytometry investigation system 302, and one or more computer memory of the at least one computer memory 1354 and/or one or more computer processor of the at least one processor 1352 can be located outside of such a common enclosure and can be located locally or remotely to the flow cytometry investigation system 1302. Communication connections between any componentry of the flow cytometry system (e.g., between the data evaluation and control system 1350 and the flow cytometry investigation system 302, or between components of the data evaluation system) can be wired connections or wireless connections or a combination of wired and wireless connections.

Aspects of the present disclosure are further disclosed and exemplified with the following examples.

### Example 1

Tests are performed using the Virus Counter® 3100 flow cytometer. The flow cytometer has a flow cell with an investigation channel having a square cross-sectional shape with side dimension of 250 micrometers. The flow cytometer was set for a fluid sample flow rate of 300 nanoliters per minute and a sheath fluid flow rate of 350 microliters per minute. Excitation is provided by a laser having a focal area width of about 10 micrometers in the direction of flow. Based on these design and operating characteristics, it is estimated that the photomultiplier tubes will make about 16-17 measurements of nanoparticles in the core stream of the sample flow passing through the focal area of the laser.

Fluid samples are prepared from aqueous buffered solution with added 120 nanometer polystyrene beads at a concentration of about 8×107 particles per milliliter. The polystyrene beads contain fluorescent labels providing a range of fluorescent emission wavelengths, including wavelengths corresponding to the two detection channels of the flow cytometer. The concentration of the beads is near the upper limit of detection of the flow cytometer, which is considered to be about 1×10⁸ particles per milliliter with less than 2% particle coincidence.

Flow cytometry runs are performed on both the fluid samples with the beads and blank fluid samples of only the clean buffered solution. Fluorescent response data is obtained from both detection channels.

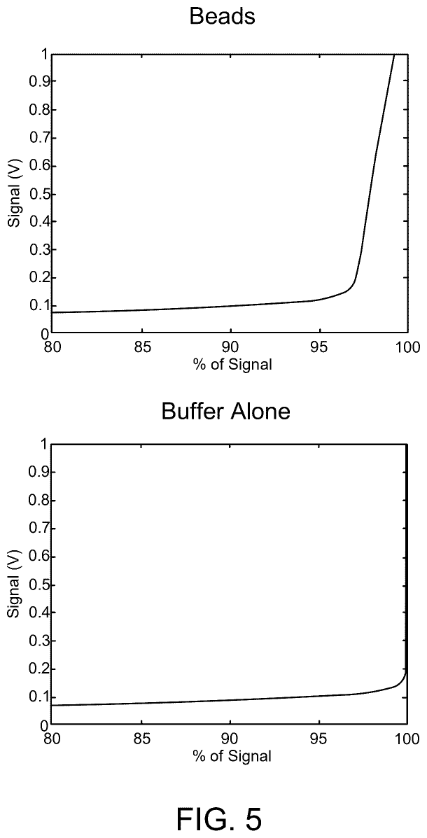

FIG. 5 shows a plot of fluorescent response data from the fluid samples with the beads (upper plot) and a plot of the blank flow cytometry data from the blank fluid samples of clean buffered solution obtained by the first fluorescence detection channel. Both plots show signal magnitude (in volts) for the largest 20 percent (top two deciles) data value magnitudes plotted to show magnitude distribution from smallest to largest (similar to Plot B of FIG. 4). As seen in both plots of FIG. 5, the data for the bead-containing samples and the blank fluid samples have similar profiles, with most of the data value magnitudes for both plots being relatively small except for the largest few percentiles.

The blank flow cytometry data collected through the first detector channel was evaluated for setting a minimum signal threshold requirement and minimum peak width requirement as an event condition for identifying particle detection in the bead-containing sample data from the first detector channel. To do that, the largest 10 percent of background signal data values are removed from the blank flow cytometry data (removal of all of the top decile), and the remaining 90 percent of the background signal data values (the selected portion) were averaged to establish a background baseline level of 0.044 volts (average signal value of the 90 percent selected portion of the background signal data values). The standard deviation of the selected portion of the background signal data values is determined to be 0.034. Various combinations of minimum signal threshold requirements and minimum peak width requirements were considered for the event condition, and each event condition combination was applied for analysis of the blank flow cytometry data (including all background signal data values, not just the 90% selected portion) and the number of resulting detection events (false positives) was recorded. The minimum signal threshold requirement (MSTR) was determined as the background baseline level (BL) plus the standard deviation (SD) multiplied by a multiplier factor (n) according to the following equation:

$$MSTR = BL + n * BL$$

The minimum peak width requirement was a minimum number of consecutive data signal values above the minimum signal threshold required to identify a temporal peak as a detection event.

Results are summarized in Table 1.

TABLE 1

| | | | Multiplier | Minimum | | Number of |
|---|---|---|---|---|---|---|
| Event | Background | | Of | Signal | Minimum | Detection |
| Condition | Baseline | Standard | Standard | Threshold | Peak | Events |
| Combination | Level | Deviation | Deviation | Requirement | Width | (False |
| No. | (Volt) | (Volt) | (n) | (Volt) | Requirement | Positives) |
| 1 | 0.044 | 0.034 | 3 | 0.147 | 2 | 13936 |
| 2 | 0.044 | 0.034 | 3 | 0.147 | 4 | 800 |
| 3 | 0.044 | 0.034 | 3 | 0.147 | 6 | 15 |
| 4 | 0.044 | 0.034 | 2 | 0.113 | 2 | 98297 |
| 5 | 0.044 | 0.034 | 2 | 0.113 | 4 | 13976 |
| 6 | 0.044 | 0.034 | 2 | 0.113 | 6 | 1119 |
| 7 | 0.044 | 0.034 | 2 | 0.113 | 8 | 134 |
| 8 | 0.044 | 0.034 | 2 | 0.113 | 10 | 20 |

As seen in Table 1, the best combinations of minimum signal threshold requirement and minimum peak width requirement for setting an event condition are combination numbers 3 and 8, which each resulted in only a small number of detection events (false positives) when applied to the blank flow cytometry data. Particularly striking in Table 1 is that it is clear that setting an event condition based solely on a minimum signal threshold requirement would lead either to setting the threshold at a level where the number of false positives is large or if the threshold was increased to reduce the number of false positives to only a small number, the threshold could result in significant undercounting of particles when applied to fluorescent response data for a stained fluid sample with particles.

Each of detection event combination numbers 1-8 of Table 1 were then applied to analyze the fluorescent response data from flow cytometry investigation of the fluid samples with the beads and count the number of detection events. Results are summarized in Table 2.

TABLE 2

| | | Difference From |
|---|---|---|
| Event | | Blank Sample |
| Condition | Number of | Detection Events |
| Combination | Detection | (Normalized |
| No. | Events | Counts) |
| 1 | 38424 | 24488 |
| 2 | 24464 | 23664 |
| 3 | 23399 | 23384 |
| 4 | 125518 | 27221 |
| 5 | 39446 | 25470 |
| 6 | 24850 | 23731 |
| 7 | 23540 | 23406 |
| 8 | 23262 | 23242 |

Table 2 shows both the absolute number of detection events for the bead-containing sample for each event condition combination and the difference in number of detection events relative to the detection events (false positives) recorded for the corresponding blank flow cytometry data analysis. As seen in table 2, there is a wide disparity in the absolute number of detection events between the different combination numbers, which disparity can be somewhat normalized by subtracting the corresponding blank sample detection events. As expected, the event condition combinations having the highest number of detection events for the blank sample also have the highest absolute number of detection events for the bead-containing samples. However, even when normalized by subtraction of counts of blank sample detection events, the combinations with higher numbers of blank sample detection events still tend to significantly overcount detection events relative to event combinations having the lowest numbers of blank sample detection events (combinations numbers 3 and 8). The benefit of appropriately selecting a combination of minimum signal threshold requirement and minimum peak width requirement to provide a very low, positive number of blank sample detection events is thus clear. Also, the absolute and normalized counts between combination numbers 3 and 8 are extremely close, indicating either is a good choice for a detection condition, and the normalized counts of each are so close to the absolute counts that correction of flow cytometry results by subtraction of counts of blank sample detection events practically becomes unnecessary to obtain accurate and useful flow cytometry results on stained fluid samples. A common current practice is to make blank sample flow cytometry runs corresponding to stained sample flow cytometry runs and to deduct blank sample counts to obtain more useful flow cytometry results. With the present technique, flow cytometry evaluation of stained fluid samples can be simplified relative to that common practice by reducing the number of or even eliminating duplicate runs of blank fluid samples used for correction of flow cytometry results, significantly reducing the number of flow cytometry runs necessary to generate accurate and useful results.

### Example 2

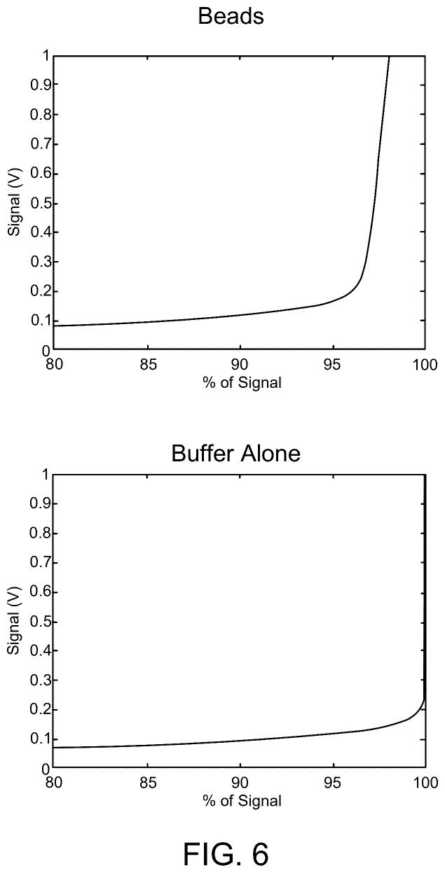

For the blank sample flow cytometry runs and bead-containing sample flow cytometry runs of Example 1, FIG. 6 shows a plot of fluorescent response data from the fluid samples with the beads (upper plot) and a plot of the blank flow cytometry data from the blank fluid samples of clean buffered solution obtained by the second fluorescence detection channel. Both plots show signal magnitude (in volts) for the largest 20 percent (top two deciles) data value magnitudes plotted to show magnitude distribution from smallest to largest, similar to as discussed for Example 1. As seen in both plots of FIG. 6, the data for the bead-containing samples and the blank fluid samples have generally similar distribution profiles, with most of the data value magnitudes for both plots being relatively small except for the largest few percentiles.

The blank flow cytometry data collected through the second detector channel was evaluated for setting a minimum signal threshold requirement and minimum peak width requirement as an event condition for identifying particle detection in the bead-containing sample data from the second detector channel. To do that, the largest 10 percent of background signal data values are removed from the blank flow cytometry data (removal of all of the top decile), and the remaining 90 percent of the background signal data values (the selected portion) were averaged to establish a background baseline level of 0.034 volt. The standard deviation of the selected portion of the background signal data values is determined to be 0.032. Similar to the process of Example 1, various combinations of minimum signal threshold requirements and minimum peak width requirements were considered for the event condition to apply to flow cytometry fluorescent response data from the second detector channel, and each considered event condition combination was applied for analysis of the blank flow cytometry data (including all background signal data values, not just the 90% selected portion) and the number of resulting detection events (false positives) was recorded. Results are summarized in Table 3.

TABLE 3

| | | | Multiplier | Minimum | | Number of |
|---|---|---|---|---|---|---|
| Event | Background | | Of | Signal | Minimum | Detection |
| Condition | Baseline | Standard | Standard | Threshold | Peak | Events |
| Combination | Level | Deviation | Deviation | Requirement | Width | (False |
| No. | (Volt) | (Volt) | (n) | (Volt) | Requirement | Positives) |
| 1 | 0.034 | 0.032 | 2 | 0.098 | 10 | 4565 |
| 2 | 0.034 | 0.032 | 3 | 0.130 | 10 | 405 |
| 3 | 0.034 | 0.032 | 4 | 0.162 | 10 | 25 |

As seen in Table 3, the best considered combination of minimum signal threshold requirement and minimum peak width requirement for setting an event condition is combination number 3, which resulted in only 25 detection events (false positives) when applied to the blank flow cytometry data from the second detector channel.

Each of detection event combination numbers 1-3 of Table 3 were then applied to analyze the fluorescent response data of the second detection channel from flow cytometry investigation of the fluid samples with the beads and count the number of detection events. Results are summarized in Table 4.

TABLE 4

| | | Difference From |
|---|---|---|
| Event | | Blank Sample |
| Condition | Number of | Detection Events |
| Combination | Detection | (Normalized |
| No. | Events | Counts) |
| 1 | 29068 | 24503 |
| 2 | 23381 | 22976 |
| 3 | 22728 | 22723 |

Table 2 shows both the absolute number of detection events for the bead-containing sample for each event condition combination and the difference in number of detection events relative to the detection events (false positives) recorded for the corresponding blank sample analysis. As seen in table 4, and similar to the results for the first detection channel summarized in Table 2, even after normalization of results by subtraction of corresponding blank sample detection event (false positive) counts, event condition combinations with higher numbers of blank sample counts also had higher numbers of normalized counts for the bead-containing samples. Also, the detection event counts for the bead-containing samples from the second detection channel are similar in total and normalized count to the results obtained from data from the first detection channel (only about 2% difference) between combination number 8 of Example 1 and combination number 3 of Example 2).

### Example Implementation Combinations.

Some nonlimiting contemplated examples of technical combinations for use with implementation of various aspects of this disclosure, with or without additional features as disclosed above or elsewhere herein, are summarized below. Also, although the features of the example combinations are illustrated with various combinations of the features, these various combinations are only exemplary, and any of the features can be combined in alternative combinations with any other feature or features, including any feature of features disclosed elsewhere herein in the written description (including the claims and abstract) and/or in the drawings.

### Methods for Evaluating Background Signal Data & Setting Requirements for a Nanoparticle Detection Evaluation Condition.

1. A method for evaluating blank flow cytometry data for setting at least one requirement of, and optionally all requirements of, an event condition for identifying as detection events temporal peaks in fluorescent response data from flow cytometry investigation of a stained fluid sample, containing a sample of material (optionally being or including biological material) for evaluation for the presence of nanoparticles (optionally being or including biological nanoparticles), for stained nanoparticles (optionally being or including stained biological nanoparticles) comprising the nanoparticles stained with a fluorescent stain providing a fluorescent response to excitation radiation during the flow cytometry investigation, the method comprising:
analyzing a portion of blank flow cytometry data from blank sample flow cytometry investigation with the excitation radiation of at least one blank fluid sample not including (being in the absence of) the stained nanoparticles, the blank flow cytometry data comprising background signal data values corresponding to detected background signal magnitudes from detection for the fluorescent response during the blank sample flow cytometry investigation; and
the portion of the blank flow cytometry data not including (being in the absence of) an excluded portion of the background signal data values, wherein the excluded portion comprises some or all of a largest 10 percent (top decile) of the background signal data values.

2. A method for determining flow cytometry background signal characteristics for setting at least one requirement of, and optionally all requirements of, an event condition for identifying as detection events temporal peaks in fluorescent response data from flow cytometry investigation of a stained fluid sample, containing a sample of a material (optionally being or including biological material) for evaluation for the presence of nanoparticles (optionally being or including biological nanoparticles), for stained nanoparticles (optionally being or including stained biological particles) comprising the nanoparticles stained with a fluorescent stain providing a fluorescent response to excitation radiation during the flow cytometry investigation, the method comprising:
determining characteristics of a portion of blank flow cytometry data from blank sample flow cytometry investigation with the excitation radiation of at least one blank fluid sample not including (being in the absence of) the material for evaluation, the blank flow cytometry data comprising background signal data values corresponding to detected background signal magnitudes from detection for the fluorescent response during the blank sample flow cytometry investigation; and
the portion of the blank flow cytometry data not including (being in the absence of) an excluded portion of the background signal data values, wherein the excluded portion comprises some or all of a largest 10 percent (top decile) of the background signal data values.

3. A method for setting at least one requirement of, and optionally all requirements of, an event condition for identifying as detection events temporal peaks in fluorescent response data from flow cytometry investigation of a stained fluid sample, containing a sample of material (optionally being or including biological material) for evaluation for the presence of nanoparticles (optionally being or including biological nanoparticles), for stained nanoparticles (optionally being or including stained biological nanoparticles) comprising the nanoparticles stained with a fluorescent stain providing a fluorescent response to excitation radiation during the flow cytometry investigation, the event condition comprising a minimum signal threshold requirement and the method comprises:
setting the minimum signal threshold requirement based on characteristics of a portion of blank flow cytometry data from blank sample flow cytometry investigation with the excitation radiation of at least one blank fluid sample not including (being in the absence of) the material for evaluation (and optionally being in the absence of any biological material), the blank flow cytometry data comprising background signal data values corresponding to detected background signal magnitudes from detection for the fluorescent response during the blank sample flow cytometry investigation; and
the portion of the blank flow cytometry data not including (being in the absence of) an excluded portion of the background signal data values, wherein the excluded portion comprises some or all of a largest 10 percent (top decile) of the background signal data values.

3.1. The method of any one of combinations 1-3, wherein the event condition comprises a minimum signal threshold requirement for fluorescent response data values in the fluorescent response data and the method comprises setting the minimum signal threshold requirement based on characteristics of the portion of the blank flow cytometry data.

3.2. The method of any one of combinations 1-3, wherein the event condition comprises a minimum peak width requirement for a temporal width of the temporal peak and the method comprises setting the minimum peak width requirement based on characteristics of the portion of the blank flow cytometry data.

3.2.1. The method of combination 3.2, wherein the minimum peak width requirement comprises a plurality of fluorescent data values of the fluorescent response data satisfying a minimum signal threshold requirement.

3.3. The method of any one of combinations 1-3, wherein the event condition comprises a minimum signal threshold requirement for fluorescent response data values in the fluorescent response data and a minimum peak width requirement for a temporal width of the temporal peak, and the method comprises:
setting the minimum signal threshold requirement and the minimum peak width requirement based on characteristics of the portion of the blank flow cytometry data.

3.3.1. The method of combination 3.3, wherein the minimum peak width requirement comprises a plurality of the fluorescent response data values each satisfying the minimum signal threshold requirement.

4. A method for setting at least one requirement of, and optionally all requirements of, an event condition for identifying as detection events temporal peaks in flow cytometry fluorescent response data from flow cytometry investigation of a stained fluid sample, containing a sample of material (optionally being or including biological material) for evaluation for presence of nanoparticles (optionally being or including stained biological nanoparticles), for stained nanoparticles (optionally being or including stained biological particles) comprising the nanoparticles stained with a fluorescent stain providing a fluorescent response to excitation radiation during the flow cytometry investigation and the fluorescent response data comprising a time series of fluorescent response data values corresponding to detected magnitudes for the fluorescent response during the flow cytometry investigation, the method comprising:
analyzing blank flow cytometry data from blank sample flow cytometry investigation with the excitation radiation of at least one blank fluid sample not including (being in the absence of) the material for evaluation (optionally in the absence of any biological material), the blank flow cytometry data comprising background signal data values corresponding to detected background signal magnitudes from detection for the fluorescent response during the blank sample flow cytometry investigation; and
setting the requirements of the event condition to include, based on the analyzing:
a minimum signal threshold requirement for the fluorescent response data values; and
a minimum peak width requirement for a temporal width of the temporal peak, the minimum peak width requirement comprising a plurality of the fluorescent response data values each satisfying the minimum signal threshold requirement.

4.1. The method of either one of combination 3.3.1 or 4, comprising setting a combination of the minimum signal threshold requirement and the minimum peak width requirement to satisfy a background condition.

4.1.1. The method of combination 4.1, wherein the background condition comprises a time series of the background signal data values from the blank sample flow cytometry investigation having a flow-normalized quantity (Q) of no more than 1×10⁶ temporal peaks per milliliter per minute of blank fluid sample flow satisfying the event condition, wherein the flow-normalized quantity is calculated as Q=N/(V*T), where Q is the flow-normalized quantity, N is a number of the temporal peaks in the time series of the background signal data values satisfying the event condition in the time series of the background signal data values, V is a volume in milliliters of the at least one blank fluid sample of the blank sample flow cytometry investigation corresponding to the time series of background signal data values and T is an elapsed time in minutes of the time series of the background signal data values.

4.1.2. The method of combination 4.1.1, wherein the flow-normalized quantity Q is no larger than 1.5×10⁵ temporal peaks per milliliter per minute of the blank fluid sample flow satisfying the event condition in the time series of the background signal data values.

4.1.3. The method of combination 4.1.1, wherein the flow-normalized quantity Q is no larger than 1.0×10⁵ temporal peaks per milliliter per minute of the blank fluid sample flow satisfying the event condition in the time series of the background signal data values.

4.1.4. The method of any one of combinations 4.1.1-4.1.3, wherein the flow-normalized quantity Q is at least as large as 1.0×10⁴ temporal peaks per milliliter per minute of the blank fluid sample flow satisfying the event condition in the time series of the background signal data values.

4.1.5. The method of any one of combinations 4.1.1-4.1.4, comprising, after setting the combination of the minimum signal threshold requirement and the minimum peak width requirement to satisfy the background condition for a flow cytometer and after performing a said flow cytometry investigation of the stained fluid sample on the flow cytometer following setting the combination, performing a background quality control check, wherein the background quality control check comprises:
performing a different blank sample flow cytometry investigation, different than the blank sample flow cytometry investigation used to set the combination, to generate different blank flow cytometry data; and
evaluating the different blank flow cytometry data relative to the background condition to identify detection events in the different blank flow cytometry data based on the detection event including the set combination of the minimum signal threshold requirement and the minimum peak width requirement; and
issuing a background data warning notification responsive to the identified detection events in the different blank flow cytometry data being larger than the flow-normalized quantity (Q) times a factor greater than 1.

4.1.6. The method of combination 4.1.5, wherein the factor is at least 1.5.

4.1.7. The method of combination 4.1.5, wherein the factor is at least 2.

4.1.8. The method of any one of combinations 4.1.5-4.1.7, wherein the factor is not larger than 4.

4.1.9. The method of any one of combinations 4.1.5-4.1.7, wherein the factor is not larger than 3.

4.2. The method of any one of combinations 4.1-4.1.9, wherein the background condition comprises at least 1 temporal peak in a time series of the background signal data values satisfying the event condition.

4.2.1 The method of combination 4.2, wherein the background condition comprises at least 2 temporal peaks in the time series of the background signal data values satisfying the event condition.

4.2.2 The method of combination 4.2, wherein the background condition comprises at least 3 temporal peaks in the time series of the background signal data values satisfying the event condition.

4.2.3 The method of combination 4.2, wherein the background condition comprises at least 4 temporal peaks in the time series of the background signal data values satisfying the event condition.

4.2.4 The method of combination 4.2, wherein the background condition comprises at least 6 temporal peaks in the time series of the background signal data values satisfying the event condition.

4.3. The method of any one of combinations 1-4.2.4, wherein the background signal data values correspond to the detected background signal magnitudes at a measurement frequency of at least 1 kilohertz (kHz).

4.4. The method of any one of combinations 1-4.3, wherein the background signal data values correspond to the detected background signal magnitudes at a measurement frequency not greater than 10 megahertz (MHz).

### Methods for Evaluating Stained Fluid Samples Using a Nanoparticle Detection Event Condition.

5. A method for evaluating a stained fluid sample, containing a sample of material (optionally being or including biological material) for evaluation for the presence of nanoparticles (optionally being or including biological nanoparticles), for stained nanoparticles (optionally being or including stained biological nanoparticles) comprising the nanoparticles stained with a fluorescent stain providing a fluorescent response to excitation radiation, the method comprising:
analyzing fluorescent response data from a flow cytometry investigation of the stained fluid sample with the excitation radiation, the fluorescent response data comprising a time series of fluorescent response data values corresponding to detected magnitudes for the fluorescent response during the flow cytometry investigation; and
the analyzing the fluorescent response data comprising identifying as detection events in the time series of fluorescent response data values temporal peaks each satisfying an event condition, wherein the event condition comprises a minimum peak width requirement for a temporal width of the temporal peak and the minimum peak width requirement includes a plurality of the fluorescent response data values each satisfying a minimum signal threshold requirement.

6. A method for evaluating a stained fluid sample, containing a sample of material (optionally being or including biological material) for evaluation for the presence of nanoparticles (optionally being or including biological nanoparticles), for stained nanoparticles (optionally being or including stained biological nanoparticles) comprising the nanoparticles stained with a fluorescent stain providing a fluorescent response to excitation radiation, the method comprising:
analyzing fluorescent response data from a flow cytometry investigation of the stained fluid sample with the excitation radiation, the fluorescent response data comprising a time series of fluorescent response data values corresponding to detected magnitudes for the fluorescent response during the flow cytometry investigation; and
the analyzing the fluorescent response data comprising identifying as detection events in the time series of fluorescent response data values temporal peaks each satisfying an event condition, wherein the event condition comprises at least one, and preferably a plurality, of the fluorescent response data values satisfying a minimum signal threshold requirement;
and wherein:
the minimum signal threshold requirement is based on characteristics of a portion of blank flow cytometry data from blank sample flow cytometry investigation with the excitation radiation of at least one blank fluid sample not including (being in the absence of) the stained nanoparticles, the blank flow cytometry data comprising background signal data values corresponding to detected signal magnitudes from detection for the fluorescent response during the blank sample flow cytometry investigation; and
the portion of the blank flow cytometry data not including (being in the absence of) an excluded portion of the background signal data values, wherein the excluded portion comprises some or all of a largest 10 percent (top decile) of the background signal data values.

6.1. The method of either one of combination 5 or combination 6, wherein the event condition comprises a minimum signal threshold requirement for fluorescent response data values in the fluorescent response data.

6.2. The method of either one of combination 5 or combination 6, wherein the event condition comprises a minimum peak width requirement for a temporal width of the temporal peak.

6.2.1. The method of combination 6.2, wherein the minimum peak width requirement comprises a plurality of fluorescent data values of the fluorescent response data satisfying a minimum signal threshold requirement.

6.3. The method of either one of combination 5 or combination 6, wherein the event condition comprises a minimum signal threshold requirement for fluorescent response data values in the fluorescent response data and a minimum peak width requirement for a temporal width of the temporal peak.

6.3.1. The method of combination 6.3, wherein the minimum peak width requirement comprises a plurality of the fluorescent response data values each satisfying the minimum signal threshold requirement.

6.4. The method of any one of combinations 5-6.3.1, comprising, prior to the analyzing fluorescent response data:
performing the method of any one of combinations 1-4.4.

### Nanoparticle Detection Event Condition Features.

7. The method of any one of combinations 1-6.4, wherein the event condition comprises a plurality of the fluorescent response data values each satisfying the minimum signal threshold requirement, and optionally with the event condition comprising a minimum peak width requirement for a temporal width of the temporal peak and the minimum peak width requirement includes a plurality of the fluorescent response data values, wherein the plurality of the fluorescent response data values comprise a minimum plurality number of the fluorescent response data values.

7.1. The method of combination 7, wherein the minimum plurality number of the fluorescent response data values are a minimum plurality number of consecutive ones of the fluorescent response data values.

7.2. The method of either one of combination 7 or combination 7.1, wherein the minimum plurality number is at least 2.

7.3. The method of combination 7.2, wherein the minimum plurality number is at least 3.

7.4. The method of combination 7.2, wherein the minimum plurality number is at least 4.

7.5. The method of combination 7.2, wherein the minimum plurality number is at least 6.

7.6. The method of combination 7.2, wherein the minimum plurality number is at least 8.

7.7. The method of combination 7.2, wherein the minimum plurality number is at least 10.

7.8. The method of any one of combinations 7-7.7, wherein the minimum plurality number is not larger than 1000.

7.9. The method of any one of combinations 7-7.7, wherein the minimum plurality number is not larger than 100.

7.10. The method of any one of combinations 7-7.7, wherein the minimum plurality number is not larger than 50.

7.11. The method of any one of combinations 7-7.7, wherein the minimum plurality number is not larger than 25.

### Blank Sample Flow Cytometry Investigation And Blank Flow Cytometry Data Analysis.

8. The method of any one of combinations 1-7.11, comprising the blank flow cytometry data from blank sample flow cytometry investigation.

8.1. The method of combination 8, comprising performing the blank sample flow cytometry investigation.

8.2. The method of either one of combination 8 or combination 8.1, wherein the blank sample flow cytometry investigation comprises a plurality of flow cytometry runs on a plurality of the blank fluid samples wherein the blank flow cytometry data includes the background signal data values from the plurality of the flow cytometry runs.

8.3. The method of any one of combinations 8-8.2, wherein the blank fluid sample is in the absence of the fluorescent stain.

8.4. The method of combination 8.3, wherein the blank fluid sample is in the absence of any fluorescent stain.

8.5 The method of combination 8.4, wherein the blank fluid sample is a buffer-only fluid sample.

8.6 The method of combination 8.5, wherein the buffer-only fluid sample comprises an aqueous buffered solution in the absence of any fluorescent stain and any sample of material for evaluation for the presence of nanoparticles, and preferably is in the absence of any of the nanoparticles.

8.7. The method of any one of combinations 8-8.2, wherein the blank fluid sample includes the fluorescent stain.

8.8. The method of combination 8.7, wherein the blank fluid sample is a reagent-only fluid sample.

8.9. The method of any one of combinations 8-8.8, wherein the blank fluid sample is in the absence of any biological nanoparticles.

8.10. The method of any one of combinations 8-8.9, wherein the blank fluid sample is in the absence of any biological material.

8.11. The method of any one of combinations 8-8.2, 8.9 and 8.10, wherein the blank fluid sample consists essentially of, or consists of, aqueous buffered solution.

8.12. The method of any one of combinations 8-8.11, comprising the excluded portion of the background signal data values of any one of combinations 1-3 and 6

9. The method of combination 8.12, wherein the excluded portion of blank flow cytometry data includes at least 80 percent, preferably at least 85 percent, more preferably at least 90 percent, even more preferably at least 95 percent, still more preferably at least 99 percent, and most preferably all, of the background signal data values with a largest 10 percent (top decile) of background signal data values.

9.1. The method of either one of combination 8.12 or combination 9, wherein the excluded portion of the blank flow cytometry data includes at least 80 percent, preferably at least 85 percent, more preferably at least 90 percent, even more preferably at least 95 percent, still more preferably at least 99 percent, and most preferably all, of the background signal data values with a largest 1 percent of background signal data values, or optionally with a largest 3 percent of the background signal data values, or further optionally with a largest 5 percent of the background signal data values.

9.1.1 The method of any one of combinations 8.12-9.1, wherein the excluded portion does not include (is in the absence of) at least 80 percent, preferably at least 85 percent, more preferably at least 90 percent, even more preferably at least 95 percent, still more preferably at least 99 percent, and most preferably all, of the background signal data values with a smallest 50 percent of background signal data values, or optionally with a smallest 60 percent of the background signal data values, or further optionally with a smallest 70 percent, or even optionally with a smallest 80 percent of the background signal data values.

9.2. The method of any one of combinations 8.12-9.1.1, wherein the excluded portion of the blank flow cytometry data includes some or all of a second population of the background signal data values with a second largest 10 percent (ninth decile) of the background signal data values.

9.3. The method of combination 9.2 wherein the excluded portion of the background signal data values includes at least 50 percent, preferably at least 70 percent, more preferably at least 90 percent, even more preferably at least 95 percent, still more preferably at least 99 percent, and most preferably all, of the background signal data values with the second largest 10 percent (ninth decile) of background signal data values.

10. The method of any one of combinations 8.12-9.3, wherein:
the event condition comprises the minimum signal threshold requirement;
the portion of the blank flow cytometry data has a background signal level, and
optionally the method comprises determining the background signal level for the portion of the blank flow cytometry data; and
the minimum signal threshold requirement is larger than the background signal level.

10.1. The method of combination 10, wherein the background signal level is an average of the background signal data values of the portion of the blank flow cytometry data.

10.2. The method of either one of combination 10 or combination 10.1, wherein the minimum signal threshold requirement is larger than the background signal level by an amount of at least 1.0 times a standard deviation value, preferably at least 1.5 times a standard deviation value, more preferably at least 1.8 times a standard deviation value, and even more preferably at least 2.0 times a standard deviation value, of the background signal data values of the portion of background signal data values.

10.3. The method of any one of combinations 10-10.2, wherein the minimum signal threshold requirement is larger than the background signal level by an amount of no larger than 4.0 times a standard deviation value, preferably no larger than 3.0 times a standard deviation value, more preferably no larger than 2.5 times a standard deviation value, and even more preferably no larger than 2.3 times a standard deviation value of the background signal data values of the portion of background signal data values; and
wherein in one preferred implementation the minimum signal threshold requirement is larger than the background signal level by an amount within a range of from 1.8 to 3.0, and optionally from 1.8 to 2.5, times a standard deviation value of the background signal data values of the portion of background signal data values.

### Some Combinations Including Flow Cytometry Investigation of a Stained Fluid Sample.

11. The method of any one of combinations 1-10.3, comprising performing the flow cytometry investigation of the stained fluid sample.

11.1. The method of combination 11, comprising performing the flow cytometry investigation of the stained fluid sample after performing the method of any one of combinations 1-4.4.

11.2. The method of any one of combinations 1-11.1, comprising;
performing the method of either one of combination 5 or combination 6; and
performing the flow cytometry investigation of the stained fluid sample prior to the analyzing the fluorescent response data.

11.3. The method of any one of combinations 1-11.2, wherein the flow cytometry investigation of the stained fluid sample comprises:
flowing the stained fluid sample through an investigation channel and in the investigation channel subjecting a flow of the stained fluid sample to the excitation radiation to produce the fluorescent response from the fluorescent stain and detecting, by a detector, for radiation corresponding to the fluorescent response from the investigation channel;
generating the flow cytometry fluorescent response data comprising the time series of fluorescent response data values, the generating the flow cytometry response data comprising obtaining a series of measurements of the radiation corresponding to the fluorescent response detected by the detector.

11.4. The method of any combination 11.3, wherein the detecting comprises a measurement frequency of the detector of at least 1 kilohertz (kHz).

11.5. The method of either one of combination 11.3 or 11.4, wherein the detecting comprises a measurement frequency of the detector of not greater than 10 megahertz (MHz).

11.5.1 The method of any one of combinations 1-11.5, wherein the flow cytometry investigation of the stained fluid sample is in the absence of light scatter detection.

11.5.2 The method of any one of combinations 1-11.5.1, wherein the flow cytometry investigation of the stained fluid sample comprises detecting only for fluorescent emissions from the investigation channel.

11.6. The method of any one of combinations 1-11.5.2, wherein the flow cytometry investigation of the stained fluid sample comprises:
hydrodynamically focusing a flow of the stained fluid sample with a sheath fluid to prepare a hydrodynamically-focused flow of the stained fluid sample;
and wherein:
the flowing the stained fluid sample through the investigation channel comprises passing the hydrodynamically-focused flow of the stained fluid sample and sheath fluid through the investigation channel; and
the subjecting a flow of the stained fluid sample to the excitation radiation comprises subjecting the hydrodynamically-focused flow of the stained fluid sample to the excitation radiation;
11.7. The method of combination 11.6, wherein:
the hydrodynamically-focused flow of the stained fluid sample is in a core stream circumferentially surrounded by the sheath fluid.

11.8. The method of combination 11.7, wherein a velocity of the hydrodynamically-focused flow of the stained fluid sample in the core stream is in a range of from 0.1 to 100 centimeters per second.

11.9. The method of either one of combination 11.7 or combination 11.8, comprising wherein the core stream has a maximum cross-dimension, transverse to a direction of flow, of no larger than 100 micrometers.

11.10. The method of combination 11.9, wherein the maximum cross-dimension of the core stream is in a range of from 1 micrometer to 100 micrometers.

11.11. The method of any one of combinations 11.6-11.10, wherein the flow cytometry investigation of the stained fluid sample comprises a ratio of a volumetric flow rate of the sheath fluid through the investigation channel to a volumetric flow rate of the stained fluid sample through the investigation channel in a range of from 10 to 100000.

11.12. The method of any one of combinations 1-11.11, wherein the flow cytometry investigation of the stained fluid sample comprises a volumetric flow rate of the stained fluid sample through the investigation channel of not larger than 300 nanoliters per minute, which volumetric flow rate of the stained fluid sample is the flow rate of the hydrodynamically-focused flow of the stained fluid sample when the stained fluid sample is subjected to hydrodynamic focusing.

11.13. The method of combination 11.12, wherein the volumetric flow rate of the stained fluid sample is not larger than 250 nanoliters per minute.

11.14. The method of combination 11.12, wherein the volumetric flow rate of the stained fluid sample is not larger than 200 nanoliters per minute.

11.15. The method of combination 11.12, wherein the volumetric flow rate of the stained fluid sample is not larger than 150 nanoliters per minute.

11.16. The method of combination 11.12, wherein the volumetric flow rate of the stained fluid sample through the investigation channel is not larger than 125 nanoliters per minute.

11.17. The method of any one of combinations 11.12-11.16, wherein the volumetric flow rate of the stained fluid sample through the investigation channel is at least 0.1 nanoliter per minute, optionally at least 1 nanoliter per minute, and further optionally at least 10 nanoliters per minute.

### Flow Cytometry Data Analysis Features

12. The method of any one of combinations 1-11.17, comprising:
analyzing fluorescent response data from a flow cytometry investigation of the stained fluid sample with the excitation radiation, the fluorescent response data comprising a time series of fluorescent response data values corresponding to detected magnitudes for the fluorescent response during the flow cytometry investigation of the stained fluid sample; and
the analyzing the fluorescent response data comprising identifying as detection events in the time series of fluorescent response data values temporal peaks each satisfying an event condition.

12.1. The method of combination 12, comprising performing the method of combination 5.

12.2. The method of combination 12, comprising performing the method of combination 6.

12.3. The method of any one of combinations 12-12.2, comprising prior to the analyzing the fluorescent response data:
performing the flow cytometry investigation of the stained fluid sample.

12.4. The method of any one of combinations 12-12.3, wherein the event condition comprises at least one, and preferably a plurality, of the fluorescent response data values satisfying a minimum signal threshold requirement.

12.5. The method of combination 12.4, wherein:
the minimum signal threshold requirement is based on characteristics of a portion of blank flow cytometry data from blank sample flow cytometry investigation of at least one blank fluid sample not including (being in the absence of) the stained nanoparticles, the blank flow cytometry data comprising background signal data values corresponding to detected signal magnitudes from detection for the fluorescent response during the blank sample flow cytometry investigation; and
the portion of the blank flow cytometry data not including (being in the absence of) an excluded portion of the background signal data values, wherein the excluded portion comprises some or all of a largest 10 percent (top decile) of the background signal data values.

12.6. The method of combination 12.5, wherein the excluded portion of the background signal data values has the features as described in any of combinations 9-9.3.

12.7. The method of any one of combinations 12.4-12.6, wherein the minimum signal threshold requirement has features as described in any of combinations 10.1-10.3.

13. The method of any one of combinations 12.4-12.7, wherein the event condition comprises a minimum peak width requirement for the temporal width of the temporal peak and the minimum peak width requirement includes a plurality of the fluorescent response data values each satisfying the minimum signal threshold requirement.

13.1. The method of combination 13, wherein the minimum peak width requirement has features as described in any of combinations 7-7.11.

14. The method of any one of combinations 12-13.1, wherein the analyzing the fluorescent response data comprises evaluating the detection events to identify as qualifying peaks one or more of the detection events with one or more peak properties satisfying a peak qualification condition

14.1. The method of combination 14, comprising comparing the one or more peak properties of each of the detection events to one or more requirements of the peak qualification condition.

14.2. The method of either one of combination 14 or 14.1, wherein the peak qualification condition comprises a maximum peak width requirement for a temporal width of the detection event, and wherein the maximum peak width requirement is larger than a minimum peak width requirement for the detection event when the event condition comprises the minimum peak width requirement.

14.3. The method of combination 14.2, wherein the analyzing the fluorescent response data comprises not counting a said detection event not satisfying the maximum peak width requirement (that is having a temporal width larger than the maximum peak width requirement) as an occurrence of the stained nanoparticle.

14.4. The method of combination 14.2, wherein the analyzing the fluorescent response data comprises identifying, and optionally counting, as occurrences of two or more than two of the stained nanoparticles a said detection event not satisfying the maximum peak width requirement.

14.5. The method of any one of combinations 14.2-14.4, wherein the maximum peak width requirement comprises a maximum consecutive number of not more than 1000 of the fluorescent response data values each satisfying the minimum signal threshold requirement.

14.6 The method of any one of combinations 14.2-14.5, wherein the maximum peak width requirement comprises a time interval for the temporal width of the detection event of not more than 100 microseconds.

14.7. The method of any one of combinations 14.2-14.6, wherein the analyzing the fluorescent response data comprises counting a number of instances of detection events not satisfying the maximum peak width requirement.

14.8. The method of any one of combinations 14.2-14.7, wherein the identifying detection events comprises issuing a data warning notification, optionally a data warning notification of possible occurrence of a coincident population of the stained nanoparticles during the flow cytometry investigation of the stained fluid sample, responsive to a threshold number of the detection events having the temporal width not satisfying the maximum peak width requirement.

14.8.1. The method of any one of combinations 14.2-14.8, wherein the maximum peak width requirement is no more than a factor (F) times an expected number (NE) of fluorescent response data values per nanoparticle in the time series of fluorescent response data values, and wherein the factor (F) is larger than one.

14.8.2. The method of combination 14.8.1, wherein NE is determined as follows:

$${N}_{E} = ( L \div v ) * f$$

where L is a length in micrometers of the investigation zone in which the stained fluid sample is subjected to the excitation radiation during the flow cytometry investigation of the stained fluid sample, vis a velocity in micrometers per second of the stained fluid sample through the investigation zone during the flow cytometry investigation of the stained fluid sample, and f is a frequency, as number per second, of the fluorescent response data values in the time series of fluorescent response data values.

14.8.3. The method of either one of combination 14.8.1 or 14.8.2, wherein the factor (F) is not larger than 2.0.

14.8.4. The method of either one of combination 14.8.1 or 14.8.2, wherein the factor (F) is not larger than 1.7.

14.8.5 The method of either one of combination 14.8.1 or 14.8.2, wherein the factor (F) is not larger than 1.5.

14.8.6. The method of either one of combination 14.8.1 or 14.8.2, wherein the factor (F) is not larger than 1.3

14.8.7. The method of any one of combinations 14.8.1-14.8.6, wherein the factor (F) is at least 1.1.

14.8.8. The method of any one of combinations 14.8.1-14.8.6, wherein the factor (F) is at least 1.15.

14.8.9. The method of any one of combinations 14.8.1-14.8.6, wherein the factor (F) is at least 1.2.

14.9. The method of any one of combinations 14.2-14.8.9, wherein the evaluation condition comprises the minimum peak width requirement.

14.10. The method of combination 14.9, wherein the maximum peak width requirement is no larger than 10.0 times the minimum peak width requirement.

14.10.1. The method of combination 14.9 wherein the maximum peak width requirement is no larger than 8.0 times the minimum peak width requirement.

14.10.2. The method of combination 14.9 wherein the maximum peak width requirement is no larger than 6.0 times the minimum peak width requirement.

14.10.3. The method of combination 14.9 wherein the maximum peak width requirement is no larger than 4.0 times the minimum peak width requirement.

14.10.4. The method of combination 14.9 wherein the maximum peak width requirement is no larger than 3.0 times the minimum peak width requirement.

14.10.5. The method of combination 14.9 wherein the maximum peak width requirement is no larger than 2.0 times the minimum peak width requirement.

14.11. The method of any one of combinations 14.9-14.10.5, wherein the maximum peak width requirement is at least 1.1 times the minimum peak width requirement

14.11.1 The method of any one of combinations 14.9-14.10.5, wherein the maximum peak width requirement is at least 1.2 times the minimum peak width requirement.

14.11.2 The method of any one of combinations 14.9-14.10.5, wherein the maximum peak width requirement is at least 1.3 times the minimum peak width requirement.

14.11.3 The method of any one of combinations 14.9-14.10.5, wherein the maximum peak width requirement is at least 1.6 times the minimum peak width requirement.

14.11.4 The method of any one of combinations 14.9-14.10.4, wherein the maximum peak width requirement is at least 2.0 times the minimum peak width requirement.

14.11.5 The method of any one of combinations 14.9-14.10.3, wherein the maximum peak width requirement is at least 3.0 times the minimum peak width requirement.

14.11.6 The method of any one of combinations 14.9-14.10.2, wherein the maximum peak width requirement is at least 4.0 times the minimum peak width requirement.

14.12. The method of any one of combinations 14-14.11.6, wherein the peak qualification condition comprises a maximum peak height requirement for a peak height of the detection event, and optionally the maximum peak height requirement is relative to the minimum signal threshold requirement of the event condition when the event condition comprises a minimum signal threshold requirement.

14.13. The method of combination 14.12, wherein the evaluating the detection events comprises not counting as an occurrence of the stained nanoparticle a said detection event not satisfying the maximum peak height requirement (that is having a peak height exceeding the maximum peak height requirement).

14.14. The method of combination 14.12 or combination 14.13, wherein the evaluating the detection events comprises counting a number of instances of the detection events not satisfying the maximum peak height requirement.

14.15. The method of any one of combinations 14.12-14.14, wherein the evaluating the detection events comprises issuing a data warning notification, optionally a data warning notification of possible occurrence of a particle swarm condition (optionally, a particle aggregation condition) for the stained nanoparticles during the flow cytometry investigation of the stained fluid sample, responsive to a threshold number of the detection events having the peak heights larger than the maximum peak height.

14.16. The method of any one of combinations 14-14.15, wherein the evaluating the detection events comprises counting a number of instances of the detection events not satisfying the peak qualification condition.

14.17. The method of any one of combinations 14-14.16, wherein the evaluating the detection events comprises issuing a data warning notification responsive to a to a threshold number of the detection events not satisfying the peak qualification condition.

14.18. The method of any one of combinations 14-14.17, wherein the evaluating the detection events comprises, for each said detection event prior to determining whether the detection event satisfies the peak qualification condition:
fitting a curve to at least a portion of the detection event, and preferably to all of the fluorescent response data values of the detection event;
and, optionally, at least one, or a plurality, and preferably all, of the one or more peak properties, evaluated relative to a requirement of the peak qualification condition for each said detection event are determined based on the fitted curve.

14.19. The method of combination 14.18, wherein at least one, optionally both, of the maximum peak width requirement and the maximum peak height requirement of any one of the preceding combinations are determined based on the fitted curve for each of the detection events.

14.20. The method of either one of combination 14.18 or combination 14.19, wherein the fitted curve of each said detection event is a curve fitted to the fluorescent response data values of the detection event having the data values at least as large as the minimum signal threshold requirement of the event condition when the event condition comprises a minimum signal threshold requirement.

14.21. The method of any one of combinations 14.18-14.20, wherein the one or more peak properties comprise a degree of fit, corresponding to a closeness of a fit of the fitted curve to the data values of the detection event, optionally a larger value for the degree of fit corresponds to a better fit of the fitted curve to the fluorescent response data values of the corresponding detection event, and further optionally the degree of fit is a value in a range of from 0 to 1 (e.g., coefficient of determination or R-squared measure) with a larger value in the range corresponding to a better fit of the fitted curve to the fluorescent response data values of the corresponding detection event.

14.22. The method of combination 14.21, wherein the peak qualification condition comprises a minimum degree of fit for the property of the degree of fit.

14.23. The method of combination 14.22, wherein the analyzing the fluorescent response data comprises not counting as an occurrence of the stained nanoparticle a said detection event having the degree of fit not satisfying the minimum degree of fit.

14.24. The method of combination 14.22, wherein the analyzing the fluorescent response data comprises identifying, and optionally counting, as occurrences of two or more than two of the stained nanoparticles a said detection event having a degree of fit not satisfying the minimum degree of fit.

14.25. The method of combination 14.22, wherein the analyzing the fluorescent response data comprises identifying, and optionally counting, as occurrences of two or more than two of the stained nanoparticles a said detection event having a degree of fit not satisfying the minimum degree of fit and having the temporal width larger than the maximum peak width requirement according to any one of the preceding combinations.

14.26. The method of any one of combinations 14.22-14.25, wherein the analyzing the fluorescent response data comprises issuing a data warning notification, optionally a data warning notification of possible occurrence of an event of particle coincidence of the stained nanoparticles during the flow cytometry investigation of the stained fluid sample, responsive to a threshold number of the detection events having the degree of fit not satisfying the minimum degree of fit, and optionally with the threshold number of the detection events also having the temporal width larger than the maximum peak width requirement.

14.27. The method of any one of combinations 14.22-14.26, wherein the analyzing the fluorescent response data comprises counting a number of instances of detection events having the degree of fit not satisfying the minimum degree of fit.

14.28. The method of any one of combinations 14.18-14.27, wherein the fitted curve corresponds to, or approximates, a normal distribution (Gaussian distribution) of points on the fitted curve.

### Determining Particle Size & Particle Populations.

15. The method of any one of combinations 14-14.28, wherein the analyzing the fluorescent response data further comprises:
for at least a portion of the qualifying peaks, determining, based on the one or more peak properties, a nanoparticle type for the stained nanoparticle occurrence corresponding to each of the qualifying peaks.

15.1. The method of combination 15, wherein the nanoparticle type comprises a nanoparticle size and the determining a nanoparticle type comprises determining the nanoparticle size.

15.2. The method of combination 15.1, wherein:
the determining a nanoparticle type comprises identifying different portions of the qualifying peaks as corresponding to different ones of multiple different populations of the stained nanoparticles based on the determined nanoparticle size, and optionally separately counting occurrences of the stained nanoparticles determined as belonging to each said different population.

15.3. The method of combination 15.1 or combination 15.2, wherein the one or more peak properties comprise a peak area, and the determining a nanoparticle size comprises correlating the peak areas of the at least a portion of the qualifying peaks to sizes of the stained nanoparticles.

### Using Multiple Fluorescent Stains.

16. The method of any one of combinations 14-15.3, wherein:
the fluorescent stain is one of a plurality of different fluorescent stains in the stained fluid sample to label different particle features, each of the different fluorescent stains of the plurality of different fluorescent stains having a different said fluorescent response during the flow cytometry investigation of the stained fluid sample responsive to a corresponding said excitation radiation, which corresponding said excitation radiation can be the same or different for different ones of the plurality of different fluorescent stains, the plurality of different fluorescent stains comprising at least a first fluorescent stain and second fluorescent stain;
the flow cytometry fluorescent response data comprises a plurality of different time series corresponding to different ones of the plurality of different fluorescent stains, wherein each of the different said time series comprises fluorescent response data values corresponding to the detected magnitudes for the corresponding different said fluorescent response of the corresponding different fluorescent stain during the flow cytometry investigation of the stained fluid sample;
each said different time series having a different corresponding said minimum signal threshold requirement, different corresponding said detection events and a different corresponding said peak qualification condition;
and the method comprises performing the evaluating the detection events for the corresponding said detection events of each of the different said time series relative to the corresponding said peak qualification condition and identifying as corresponding qualifying peaks for the each of the different said time series the corresponding said detection events satisfying the corresponding said peak qualification condition.

16.1. The method of combination 16, comprising the analyzing the fluorescent response data, wherein the analyzing the fluorescent response data comprises for each of the different fluorescent stains:
identifying in each different said time series of corresponding said fluorescent response data values as corresponding said detection events temporal peaks each satisfying a corresponding said event condition.

16.2. The method of combination 16 or combination 16.1, comprising the analyzing the fluorescent response data of combination 5, wherein the analyzing the fluorescent response data comprises for each of the different fluorescent stains:
identifying in each different said time series of corresponding said fluorescent response data values as corresponding said detection events temporal peaks each satisfying a corresponding said event condition, wherein the corresponding event condition comprises a corresponding minimum peak width requirement for a temporal width of the peak and the minimum peak width requirement includes a plurality of the corresponding said fluorescent response data values each satisfying a corresponding said minimum signal threshold requirement.

16.3 The method of combination 16.2, wherein each corresponding said minimum peak width requirement has features as described in any of combinations 7-7.11

16.4. The method of any one of combinations 16-16.3, comprising the analyzing the fluorescent response data of combination 6, and wherein the corresponding said minimum signal threshold requirement is based on a corresponding said portion of corresponding said blank flow cytometry data from the blank sample flow cytometry investigation of the at least one blank fluid sample not including (being in the absence of) the different fluorescent stain, preferably not including (being in the absence of) any one of the different fluorescent stains of the plurality of different fluorescent stains, and more preferably not including (being in the absence of) any fluorescent stains;
and wherein for each said different fluorescent stain:
the corresponding said blank flow cytometry data comprises corresponding said background signal data values corresponding to the detected signal magnitudes from detection for the different fluorescent response of the different fluorescent stain during the blank sample flow cytometry investigation;
the analyzing the fluorescent response data comprises, for the corresponding different said time series, comparing at least a portion of the fluorescent response data values of the corresponding different said time series to the corresponding said minimum signal threshold requirement; and
the corresponding said portion of the corresponding said blank flow cytometry data does not include (is in the absence of) a corresponding said excluded portion of the corresponding said background signal data values; and
the corresponding said excluded portion of the corresponding said background signal data values comprises some or all of the largest 10 percent (top decile) of the corresponding said background signal data values.

16.5 The method of combination 16.4, wherein for each of the different fluorescent stains, the corresponding said excluded portion of the corresponding said background signal data values has features as described in any of combinations 9-9.3.

16.6 The method of any one of combinations 16-16.5, wherein the minimum signal threshold requirement has features of any one of combinations 10.1-10.3.

16.7. The method of any one of combinations 16-16.6, comprising, for each of the different said fluorescent stains, prior to performing the corresponding said evaluating the corresponding said detection events, performing the method of any one of combinations 1-4.4

### Assessment of Particle Attributes From Multiple Fluorescent Stains

17. The method of any one of combinations 16-16.7, wherein the analyzing the fluorescent response data comprises:
identifying time-correlated coincidental occurrences of the corresponding said qualifying peaks of the different said time series of at least the first fluorescent stain and the second fluorescent stain; and
identifying, and optionally counting, occurrences of the stained nanoparticles stained with both the first fluorescent stain and the second fluorescent stain.

17.1. The method of combination 17, wherein the nanoparticles are biological nanoparticles.

17.2. The method of either one of combination 17 or combination 17.1, wherein the nanoparticles comprise a membrane and a lumen surrounded by the membrane, and the first fluorescent stain is a stain for a feature or features of the membrane and the second fluorescent stain is a stain for a different feature or features of the lumen.

17.3. The method of any one of combinations 17-17.2, wherein the first fluorescent stain comprises a lipophilic stain, optionally for binding to a membrane of the nanoparticles.

17.4. The method of any one of combinations 17-17.3, wherein the first fluorescent stain comprises a binding site-specific fluorescent stain, optionally a fluorescent antibody stain, or a fluorescent aptamer stain, or combinations thereof.

17.5. The method of any one of combinations 17-17.4, wherein the first fluorescent stain comprises a fluorescent antibody stain, optionally for binding with a surface protein of the nanoparticles.

17.6. The method of any one of combinations 17-17.5, wherein the second fluorescent stain comprises a nucleic acid stain.

17.7. The method of any one of combinations 17-17.6, wherein the nanoparticles comprise a cargo, optionally in a lumen of the nanoparticles, and the method comprises:
evaluating the corresponding qualifying peaks of the different said time series of the second fluorescent stain of the identified time-correlated coincidental occurrences to determine a relative loading level of the cargo in different ones of the identified occurrences of the stained nanoparticles stained with both the first fluorescent stain and the second fluorescent stain.

17.8. The method of combination 17.7, wherein the second fluorescent stain is a nucleic acid stain and the cargo is a nucleic acid-containing cargo.

17.9. The method of combination 17.8, wherein the nucleic acid-containing cargo comprises DNA, RNA, or combinations thereof.

17.10. The method of any one of combinations 17.7-17.9, wherein the cargo comprises a medical product, optionally a drug product.

17.11. The method of any one of combinations 17.7-17.10, wherein the cargo comprises a biopharmaceutical.

17.12. The method of either one of combination 17 or combination 17.1, wherein the first fluorescent stain comprises a binding site-specific fluorescent stain, optionally a fluorescent antibody stain, or a fluorescent aptamer stain, or combinations thereof.

17.13. The method of either one of combination 17 or combination 17.1, wherein the first fluorescent stain comprises a fluorescent antibody stain, optionally for binding with a surface protein.

17.14. The method of either one of combination 17.12 or combination 17.13, wherein the second fluorescent stain is a lipophilic stain, optionally for staining a lipophilic feature or features of a membrane of the nanoparticles.

17.15. The method of any one of combinations 17-17.14, comprising identifying, and optionally counting, occurrences of the stained nanoparticles stained with the first fluorescent stain and not stained by the second fluorescent stain.

17.16. The method of any one of combinations 17-17.15, comprising identifying, and optionally counting, occurrences of the stained nanoparticles stained with the second fluorescent stain and not stained with the first fluorescent stain.

### Other Features.

18. The method of any one of combinations 1-17.16, wherein the stained nanoparticles have a size in a range of from 10 nanometers to 1000 nanometers.

18.1. The method of any one of combinations 1-18, wherein the nanoparticles comprise extracellular vesicles, or lipid nanoparticles, or viral particles, or combinations thereof.

18.1.1. The method of any one of combinations 1-18.1, wherein the nanoparticles comprise virus-size particles.

18.2. The method of any one of combinations 1-18.1.1, wherein the nanoparticles comprise extracellular vesicles.

18.3. The method of combination 18.2, wherein the extracellular vesicles, and the stained extracellular vesicles of the stained nanoparticles, have a size in a range of from 10 to 1000 nanometers.

18.4. The method of any one of combinations 1-18.1.1, wherein the nanoparticles comprise lipid nanoparticles.

18.5. The method of combination 18.4, wherein the lipid nanoparticles, and the stained lipid nanoparticles of the stained nanoparticles, have a size in a range of from 10 to 1000 nanometers.

18.6. The method of any one of combinations 1-18.1.1, wherein the nanoparticles comprise viral particles.

18.7. The method of combination 18.6, wherein the viral particles, and the stained viral particles of the stained nanoparticles, have a size in a range of from 10 to 1000 nanometers.

18.7.1. The method of any one of combinations 18-18.7, wherein the stained nanoparticles have a size of at least 20 nanometers.

18.7.2. The method of any one of combinations 18-18.7, wherein the stained nanoparticles have a size of at least 25 nanometers.

18.7.3. The method of any one of combinations 18-18.7, wherein the stained nanoparticles have a size of at least 30 nanometers.

18.7.4. The method of any one of combinations 18-18.7, wherein the stained nanoparticles have a size of at least 40 nanometers.

18.7.5. The method of any one of combinations 18-18.7.4, wherein the stained nanoparticles have a size of not larger than 900 nanometers.

18.7.6. The method of any one of combinations 18-18.7.4, wherein the stained nanoparticles have a size of not larger than 800 nanometers.

18.7.7. The method of any one of combinations 18-18.7.4, wherein the stained nanoparticles have a size of not larger than 600 nanometers

18.7.8. The method of any one of combinations 18-18.7.4, wherein the stained nanoparticles have a size of not larger than 400 nanometers.

18.7.9. The method of any one of combinations 18-18.7.4, wherein the stained nanoparticles have a size of not larger than 300 nanometers.

18.7.10. The method of any one of combinations 18-18.7.4, wherein the stained nanoparticles have a size of not larger than 200 nanometers.

18.7.11. The method of any one of combinations 18-18.7.4, wherein the stained nanoparticles have a size of not larger than 100 nanometers.

18.8. The method of any one of combinations 1-18.7.11, comprising performing the method of any one of combinations 1-4.4 and 6, or the analyzing the fluorescent response data of either of combination 5 or combination 6, or performing the blank sample flow cytometry investigation of any one of combinations 1-4.4 and 6, or combinations thereof, and wherein:
the blank sample flow cytometry investigation is according to the same flow cytometry conditions as for the flow cytometry investigation of the stained fluid sample of any one of combinations 11-11.17, except using a said blank fluid sample of any one of the preceding combinations rather than a said stained fluid sample.

18.9. The method of combination 18.8, wherein the blank sample flow cytometry investigation and the flow cytometry investigation of the stained sample both use the same flow cytometer.

### Computer-Implemented Methods

19. The method of any one of combinations 1-18.9 comprising performing as a computer-implemented method:
the analyzing a portion of blank flow cytometry data of any one of combination 1 or of any other of the preceding combinations; and/or
the determining the characteristics of a portion of blank flow cytometry data method of combination 2 or of any other of the preceding combinations; and/or
the setting the minimum signal threshold requirement of combination 3 or of any other of the preceding combinations; and/or
the analyzing the blank flow cytometry data of combination 4 or of any other of the preceding combinations; and/or
the setting requirements of the event condition to include the minimum signal threshold requirement and the minimum peak width requirement of combination 4 or of any other of the preceding combinations; or
combinations thereof.

19.1. The method of claim 19, wherein the computer-implemented method comprises:
executing by at least one processor a computer program with instructions configuring the at least one processor to perform the computer-implemented method.

19.2. The method of combination 19.1, wherein the computer-implemented method comprises accessing the blank flow cytometry data by the at least one processor executing the computer program.

19.3. The method of combination 19.2, wherein the accessing the blank flow cytometry data comprises accessing the blank flow cytometry data stored in one or more computer-readable media, optionally being or comprising one or more non-transitory computer-readable media.

19.4. The method of any one of combinations 19.1-19.3, wherein the computer-implemented method comprises manipulating by the at least one processor the blank flow cytometry data according to the instructions of the computer program to determine the background signal level.

19.5. The method of any one of combinations 19-19.4, wherein the computer-implemented method comprises storing a product of the computer-implemented method in one or more computer-readable media, optionally being or comprising one or more non-transitory computer-readable media; and
optionally, the one or more computer-readable media has stored therein the blank flow cytometry data.

19.6. One or more computer-readable media, optionally being or comprising one or more non-transitory computer-readable media, storing computer-executable instructions that, when executed by at least one processor, perform the computer-implemented method of any one of combinations 19-19.5.

19.7. A method of any one of the preceding combinations comprising performing as a computer-implemented method:
the setting the minimum signal threshold requirement of combination 2, or combination 4, or of any other of the preceding combinations; and/or
the setting the minimum peak width requirement of combination 4, or any other of the preceding combinations; or combinations thereof.

19.8. The method of combination 19.7, wherein the computer-implemented method comprises:
executing by at least one processor a computer program with instructions configuring the at least one processor to perform the setting the minimum signal threshold requirement or the setting the minimum peak width requirement, or both.

19.9. The method of combination 19.8, wherein the computer-implemented method comprises accessing the blank flow cytometry data by the at least one processor executing the computer program.

19.10. The method of combination 19.9, wherein the computer-implemented method comprises accessing the blank flow cytometry data stored in one or more computer-readable media, optionally being or comprising one or more non-transitory computer-readable media.

19.11. The method of any one of combinations 19.8-19.10, wherein the computer-implemented method comprises manipulating by the at least one processor the blank flow cytometry data according to the instructions of the computer program to set the minimum signal threshold requirement value or the minimum peak width requirement, or both.

19.12. The method of any one of combinations 19.7-19.11, wherein the computer-implemented method comprises storing the minimum signal threshold requirement, or the minimum peak width requirement, or both, in one or more computer-readable media, optionally being or comprising one or more non-transitory computer-readable media; and
optionally the one or more computer-readable media has stored therein the blank flow cytometry data, or the background signal level, or combinations thereof.

19.13. One or more computer-readable media, optionally being or comprising one or more non-transitory computer-readable media, storing computer-executable instructions that, when executed by at least one processor, perform the computer-implemented method of any one of combinations 19.7-19.12; and
optionally, the one or more computer-readable media is the one or more computer-readable media of combination 19.6.

19.14. A method of any one of the preceding combinations comprising performing as a computer-implemented method the analyzing the fluorescent response data of combination 5, and/or of combination 6, and/or of any other of the preceding combinations, or combinations thereof.

19.15. The method of combination 19.14, wherein the computer-implemented method comprises:
executing by at least one processor a computer program with instructions configuring the at least one processor to perform the analyzing the fluorescent response data.

19.16. The method of combination 19.15, wherein the computer-implemented method comprises accessing the flow cytometry fluorescent response data by the at least one processor executing the computer program.

19.17. The method of combination 19.16, wherein the computer-implemented method comprises accessing the flow cytometry fluorescent response data stored in one or more computer-readable media, optionally being or comprising one or more non-transitory computer-readable media.

19.18. The method of any one of combinations 19.15-19.17, comprising manipulating by the at least one processor the flow cytometry fluorescent response data according to the instructions of the computer program to perform the analyzing the fluorescent response data.

19.19. The method of any one of combinations 19.14-19.18, comprising storing results of the analyzing the fluorescent response data in one or more computer-readable media, optionally being or comprising one or more non-transitory computer-readable media; and
optionally the one or more computer-readable media has stored therein the flow cytometry fluorescent response data.

19.20. One or more computer-readable media, optionally being or comprising one or more non-transitory computer-readable media, storing computer-executable instructions that, when executed by at least one processor, perform the analyzing the fluorescent response data of any one of the preceding combinations; and
optionally, wherein the one or more computer-readable media is the one or more computer-readable media of combination 19.6 or combination 19.13.

19.21. A method of any one of the preceding combinations comprising issuing a data warning notification, comprising displaying the data warning notification on a graphic display, optionally together with displaying results of the analyzing the fluorescent response data on the graphic display.

19.22. A method of any one of the preceding combinations comprising issuing a data warning notification, comprising storing the data warning notification in one or more computer-readable media, optionally being or comprising one or more non-transitory computer-readable media; and
optionally, the one or more computer-readable media comprises the computer-readable media of any one of combinations 19.6, 19.13 and 19.20.

19.23. The method of combination 19.22, comprising storing the data warning notification in the one or more computer-readable media together with results of the analyzing the fluorescent response data.

### System Combinations.

20. A flow cytometry data analysis system, comprising:
at least one processor;
one or more computer-readable media, optionally being or comprising one or more non-transitory computer-readable media, having stored instructions; and
a communication connection between the at least one processor and the one or more computer-readable media, the communication connection configured for the at least one processor to access the one or more computer-readable media and the stored instructions;
wherein, the stored instructions are executable by the at least one processor to configure the at least one processor to perform as a computer-implemented method a flow cytometry data analysis operation comprising:
the analyzing the fluorescent response data of any one of the preceding combinations; and/or
the analyzing a portion of blank flow cytometry data of any one of the preceding combinations; and/or
the determining characteristics of a portion of blank flow cytometry data of any other of the preceding combinations, and/or
the setting at least one requirement, and optionally all requirements of, the event condition of any one of the preceding combinations; and/or
the analyzing the blank flow cytometry data of any one of the preceding combinations; and/or
the setting the minimum signal threshold requirement of any one of the preceding combinations; and/or
the setting the minimum peak width requirement of or any one of the preceding combinations; or
combinations thereof.

20.1. The system of combination 20, wherein the flow cytometry data analysis operation comprises a computer-implemented method of any one of combinations 19-19.23.

20.2. A flow cytometry data analysis system, comprising:
at least one processor;
one or more computer-readable media, optionally being or comprising one or more non-transitory computer-readable media, having stored instructions; and
a communication connection between the at least one processor and the one or more computer-readable media, the communication connection configured for the at least one processor to access the one or more computer-readable media and the stored instructions;
wherein, the stored instructions are executable by the at least one processor to configure the at least one processor to perform a computer-implemented method of any one of combinations 19-19.23.

21. A flow cytometry system, comprising:
a flow cytometry investigation system; and
a data evaluation and control system communicatively connected to the flow cytometry investigation system and configured to control operation of the flow cytometry investigation system to perform flow cytometry investigations of fluid samples.

21.1. The flow cytometry system of combination 21, wherein the data evaluation and control system comprises:
at least one processor;
stored instructions executable by the at least one processor to control the operation of the flow cytometry investigation system to perform the flow cytometry investigations.

21.2. The flow cytometry system of combination 21.1, wherein the data evaluation and control system comprises:
one or more computer-readable media, optionally being or comprising one or more non-transitory computer-readable media, storing the stored instructions;
a communication connection between the at least one processor and the one or more computer-readable media, the communication connection configured for the at least one processor to access the one or more computer-readable media and the stored instructions.

21.3. The flow cytometry system of either one of combination 21.1 or 21.2, wherein, the stored instructions are executable by the at least one processor to configure the at least one processor to perform a computer-implemented method of any one of combinations 19-19.23.

21.4. The flow cytometry system of any one of combinations 21-21.3, wherein the data evaluation and control system comprises the flow cytometry data analysis system of any one of combinations 20-20.2.

21.5. The flow cytometry system of any one of combinations 21-21.4, wherein the flow cytometry investigation system comprises:
an investigation channel configured to conduct flow of fluid samples during flow cytometry investigations;
a radiation source configured to provide excitation radiation to the investigation channel during flow cytometry investigations;
a radiation detection system with at least one radiation detector configured to detect at least one fluorescent emission signature from the investigation channel.

21.6. The flow cytometry system of combination 21.5, wherein the radiation source comprises at least one laser.

21.7. The flow cytometry system of either one of combination 21.5 or 21.6, wherein the radiation detection system comprises at least two radiation detectors configured to detect different fluorescent emission signatures corresponding to different fluorescent stains.

21.8. The flow cytometry system of any one of combinations 21.5-21.7 configured to perform, at the control of the data evaluation and control system, the flow cytometry investigation of the stained fluid sample according to any one of combinations 11-11.17.

The foregoing description of the present invention and various aspects thereof has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described hereinabove are further intended to explain known modes of practicing the invention and to enable others skilled in the art to utilize the invention in such or other embodiments and with various modifications required by the particular application(s) or use(s) of the present invention. It is intended that the appended claims be construed to include alternative embodiments to the extent permitted by the prior art.

The description of a feature or features in a particular combination do not exclude the inclusion of an additional feature or features in a variation of the particular combination. Processing steps and sequencing are for illustration only, and such illustrations do not exclude inclusion of other steps or other sequencing of steps to an extent not necessarily incompatible. Additional steps may be included between any illustrated processing steps or before or after any illustrated processing step to an extent not necessarily incompatible.

The terms “comprising”, “containing”, “including” and “having”, and grammatical variations of those terms, are intended to be inclusive and nonlimiting in that the use of such terms indicates the presence of a stated condition or feature, but not to the exclusion of the presence also of any other condition or feature. The use of the terms “comprising”, “containing”, “including” and “having”, and grammatical variations of those terms in referring to the presence of one or more components, subcomponents or materials, also include and is intended to disclose the more specific embodiments in which the term “comprising”, “containing”, “including” or “having” (or the variation of such term) as the case may be, is replaced by any of the narrower terms “consisting essentially of” or “consisting of” or “consisting of only” (or any appropriate grammatical variation of such narrower terms). For example, a statement that something “comprises” a stated element or elements is also intended to include and disclose the more specific narrower embodiments of the thing “consisting essentially of” the stated element or elements, and the thing “consisting of” the stated element or elements. Examples of various features have been provided for purposes of illustration, and the terms “example”, “for example” and the like indicate illustrative examples that are not limiting and are not to be construed or interpreted as limiting a feature or features to any particular example. The term “at least” followed by a number (e.g., “at least one”) means that number or more than that number. The term at “at least a portion” means all or a portion that is less than all. The term “at least a part” means all or a part that is less than all.

1. A method for evaluating a stained fluid sample, containing a sample of material for evaluation for the presence of nanoparticles, for stained nanoparticles comprising the nanoparticles stained with a fluorescent stain providing a fluorescent response to excitation radiation, the method comprising:
analyzing fluorescent response data from a flow cytometry investigation of the stained fluid sample with the excitation radiation, the fluorescent response data comprising a time series of fluorescent response data values corresponding to detected magnitudes for the fluorescent response during the flow cytometry investigation of the stained fluid sample; and
the analyzing the fluorescent response data comprising identifying as detection events in the time series of fluorescent response data values temporal peaks each satisfying an event condition, wherein the event condition comprises a minimum peak width requirement for a temporal width of the temporal peak and the minimum peak width requirement includes a plurality of the fluorescent response data values each satisfying a minimum signal threshold requirement.

2. The method of claim 1, wherein the event condition comprises a plurality of at least 6 of the fluorescent response data values each satisfying the minimum signal threshold requirement.

3. The method of claim 1, comprising comparing one or more peak properties of each of the detection events to one or more requirements of a peak qualification condition; and
the peak qualification condition comprises a maximum peak width requirement for a temporal width of the detection event, and wherein the maximum peak width requirement is larger than the minimum peak width requirement.

4. The method of claim 3, wherein the analyzing the fluorescent response data comprises not counting a said detection event not satisfying the maximum peak width requirement as an occurrence of the stained nanoparticle.

5. The method of claim 3, wherein the analyzing the fluorescent response data comprises counting a number of instances of detection events not satisfying the maximum peak width requirement.

6. The method of claim 5, wherein the identifying as detection events comprises issuing a data warning notification responsive to a threshold number of the detection events having the temporal width not satisfying the maximum peak width requirement.

7. The method of claim 3, wherein the peak qualification condition comprises a maximum peak height requirement for a peak height of the detection event.

8. The method of claim 7, wherein the identifying as detection events comprises issuing a data warning notification responsive to a threshold number of the detection events having the peak heights larger than the maximum peak height.

9. The method of claim 8, wherein:
the identifying as detection events comprises, for each said detection event prior to determining whether the detection event satisfies the peak qualification condition, fitting a curve to at least a portion of the detection event;
the one or more peak properties comprise a degree of fit, corresponding to a closeness of a fit of the fitted curve to the data values of the detection event;
the peak qualification condition comprises a minimum degree of fit for the property of the degree of fit; and
the analyzing the fluorescent response data comprises issuing a data warning notification responsive to a threshold number of the detection events having the degree of fit not satisfying the minimum degree of fit.

10. The method of claim 1, comprising, prior to the analyzing the fluorescent response data, performing the flow cytometry investigation of the stained fluid sample.

11. The method of claim 1, comprising, prior to the analyzing fluorescent response data, setting at least one requirement of the event condition, wherein the event condition comprises a minimum signal threshold requirement and the method comprises;
setting the minimum signal threshold requirement based on characteristics of a portion of blank flow cytometry data from blank sample flow cytometry investigation with the excitation radiation of at least one blank fluid sample not including the material for evaluation, the blank flow cytometry data comprising background signal data values corresponding to detected background signal magnitudes from detection for the fluorescent response during the blank sample flow cytometry investigation; and
the portion of the blank flow cytometry data not including an excluded portion of the background signal data values, wherein the excluded portion comprises some or all of a largest 10 percent (top decile) of background signal data values.

12. The method of claim 11, wherein the excluded portion of blank flow cytometry data includes at least 80 percent of the background signal data values with a largest 10 percent (top decile) of background signal data values.

13. The method of claim 12, wherein:
the portion of the blank flow cytometry data has a background signal level;
the minimum signal threshold requirement is larger than the background signal level; and
the minimum signal threshold requirement is larger than the background signal level by an amount of at least 1.0 times a standard deviation value of the background signal data values of the portion of background signal data values.

14. The method of claim 13, wherein the minimum signal threshold requirement is larger than the background signal level by an amount of no larger than 4.0 time the standard deviation value of the background signal data values of the portion of background signal data values.

15. The method of claim 11, comprising performing the blank sample flow cytometry investigation.

16. The method of claim 1, comprising performing as a computer-implemented method the analyzing the fluorescent response data;
wherein the computer-implemented method comprises executing by at least one processor a computer program with instructions configuring the at least one processor to perform the analyzing the fluorescent response data.

17. (canceled)

18. A flow cytometry data analysis system, comprising:
at least one processor;
one or more computer-readable media having stored instructions; and
a communication connection between the at least one processor and the one or more computer-readable media, the communication connection configured for the at least one processor to access the one or more computer-readable media and the stored instructions;
and wherein:
the stored instructions are executable by the at least one processor to configure the at least one processor to perform as a computer-implemented method a flow cytometry data analysis operation comprising analyzing fluorescent response data from a flow cytometry investigation of a stained fluid sample with excitation radiation;
the stained fluid sample contains a sample of material for evaluation for the presence of nanoparticles stained with a fluorescent stain providing a fluorescent response to the excitation radiation;
the fluorescent response data comprises a time series of fluorescent response data values corresponding to detected magnitudes for the fluorescent response during the flow cytometry investigation of the stained fluid sample; and
the analyzing the fluorescent response data comprises identifying as detection events in the time series of fluorescent response data values temporal peaks each satisfying an event condition, wherein the event condition comprises a minimum peak width requirement for a temporal width of the temporal peak and the minimum peak width requirement includes a plurality of the fluorescent response data values each satisfying a minimum signal threshold requirement.

19. A flow cytometry system, comprising:
a flow cytometry investigation system;
a data evaluation and control system communicatively connected to the flow cytometry investigation system and configured to control operation of the flow cytometry investigation system to perform flow cytometry investigations of fluid samples, wherein the data evaluation and control system comprises:
at least one processor;
stored instructions executable by the at least one processor to control the operation of the flow cytometry investigation system to perform the flow cytometry investigations;
one or more computer-readable media storing the stored instructions; and
a communication connection between the at least one processor and the one or more computer-readable media, the communication connection configured for the at least one processor to access the one or more computer-readable media and the stored instructions; and
the stored instructions are executable by the at least one processor to configure the at least one processor to perform a computer-implemented method comprising analyzing fluorescent response data from a flow cytometry investigation of a stained fluid sample with excitation radiation;
and wherein:
the stained fluid sample contains a sample of material for evaluation for the presence of nanoparticles stained with a fluorescent stain providing a fluorescent response to the excitation radiation;
the fluorescent response data comprises a time series of fluorescent response data values corresponding to detected magnitudes for the fluorescent response during the flow cytometry investigation; and
the analyzing the fluorescent response data comprises identifying as detection events in the time series of fluorescent response data values temporal peaks each satisfying an event condition, wherein the event condition comprises a minimum peak width requirement for a temporal width of the temporal peak and the minimum peak width requirement includes a plurality of the fluorescent response data values each satisfying a minimum signal threshold requirement.

20. The flow cytometry system of claim 19, wherein the flow cytometry investigation system comprises:
an investigation channel configured to conduct flow of fluid samples during flow cytometry investigations;
a radiation source configured to provide excitation radiation to the investigation channel during flow cytometry investigations; and
a radiation detection system with at least one radiation detector configured to detect at least one fluorescent emission signature from the investigation channel.

21-23. (canceled)
